(19) United States
(12) Patent Application Publication
(10) Pub. No.: US 2003/0083292 A1
(43) Pub. Date: May 1, 2003

(54) Inhibitors of DNA methyltransferase isoforms

(76) Inventor: Alan Robert MacLeod, Montreal (CA)

Correspondence Address: KEOWN & ASSOCIATES, 500 WEST CUMMINGS PARK, SUITE 1200, WOBURN, MA, 01801, US

(21) Appl. No.: 10/144,577

(22) Filed: May 13, 2002

Related U.S. Application Data
(60) Provisional application No. 60/290,202, filed on May 11, 2001.
(60) Provisional application No. 60/290,212, filed on May 11, 2001.

(51) Int. Cl. A61K 48/00; C07H 21/04; C12N 5/02

(52) U.S. Cl. 514/44; 435/375; 536/23.2

(57) ABSTRACT

This invention relates to the inhibition of DNA MeTase expression and enzymatic activity. The invention provides methods and agents for inhibiting specific DNA MeTase isoforms by inhibiting expression at the nucleic acid level or enzymatic activity at the protein level.

### BACKGROUND OF THE INVENTION

1. Field of the Invention

This invention relates to the fields molecular biology, cell biology and cancer therapeutics.

2. Summary of the Related Art

In mammals, modification of the 5′ position of cytosine by methylation is the only known naturally occurring covalent modification of the genome. DNA methylation patterns correlate inversely with gene expression (Yeivin, A., and Razin, A. (1993) EXS 64:523). Therefore, DNA methylation has been suggested to be an epigenetic determinant of gene expression. DNA methylation is also correlated with several other cellular processes including chromatin structure (Keshet, I., et al., (1986) Cell 44:535-543; and Kass, S. U., et al., (1997) Curr. Biol., 7:157-165), genomic imprinting (Barlow, D. P. (1993) Science, 260: 309-310; and Li. E., et al., (1993) Nature 366:362-365), somatic X-chromosome inactivation in females (6), and timing of DNA replication (Shemer, R., et al. (1996) Proc. Natl. Acad. Sci. USA 93:6371-6376).

Selig et al. discloses that the DNA 5-cytosine methyltransferase (DNA MeTase) enzymes catalyze the transfer of a methyl group from S-adenosyl methionine to the 5 position of cytosine residing in the dinucleotide sequence CpG (Selig, S., et al.,. (1988) EMBO J., 7:419-426). To date, three DNA MeTases have been identified in somatic tissues of vertebrates. Adams et al. teaches that DNMT1 is the most abundant DNA MeTase in mammalian cells (Adams, R. L., et al., (1979) Biochem. Biophys. Acta 561:345-357). Glickman et al. teaches that DNMT1 preferentially methylates hemimethylated DNA as its substrate and, therefore, it is believed to be primarily responsible for maintaining methylation patterns established in development (Glickman, F. J., et al., (1997) Biochem. Biophys. Res. Comm. 230:280-284). Okano et al. suggest that the recently identified DNA MeTase enzymes, DNMT3a and DNMT3b, encode the long sought de novo methylation activities responsible for methylating previously unmethylated DNA, to generate new patterns of DNA methylation (Okano, M., et al., (1998) Nat. Genet. 19:219-20).

DNA methylation patterns are highly plastic throughout development and involve both global demethylation and de novo methylation events (for review, see Razin, A., and Cedar, H. (1993) EXS 64:343-57). Genetic experiments have demonstrated that proper regulation of DNA methylation is essential for normal mammalian development. Li et al. disclose that mice homozygous for the targeted disruption of DNMT1 (DNMT1⁻/⁻ mice) fail to maintain established DNA methylation patterns and do not survive past mid gestation (Li, E., et al., (1992) Cell 69:915-926), and similarly Okano et al. disclose that the DNMT 3b⁻/⁻ genotype produces embryo lethality in mice, whereas DNMT3a⁻/⁻ mice develop to term but become runted and die at approximately 4 weeks of age (Okano, M., et al., (1999) Cell 99:247-57).

In addition to the role DNA methylation plays in development, it is also implicated in tumorigenesis (for review, see Jones, P. A., and Laird, P. W. (1999) Nat. Genet. 21:163-167). Baylin et al. disclose that abnormal methylation patterns are observed in malignant cells, and these patterns may contribute to tumorigenesis by improper silencing of tumor suppressor genes or growth-regulatory genes (Baylin, S. B., et al., (1998) Adv. Cancer Res. 72:141-196). Szyf et al., U.S. Pat. No. 5,919,772 discloses that tumorigenicity can be reversed by reducing the expression of DNMT1. Elevated levels of DNMT3a and DNMT3b mRNA are also found in human tumors, raising a question whether they may have a role in tumorigenesis (Li, E., et al., (1992) Cell 69:915-926, Robertson, K. D., et al. (1999) Nucleic Acids Res. 27:2291-2298, and Robertson, K. D., et al., (2000) Nucleic Acids Res. 28:2108-2113).

Therefore, there remains a need to develop agents for inhibiting specific DNA MeTase isoforms. There is also a need for the development of methods for using these agents to identify and inhibit specific DNA MeTase isoforms involved in tumorigenesis.

### BRIEF SUMMARY OF THE INVENTION

The invention provides methods and agents for inhibiting specific DNA methyltransferase (DNA MeTase) isoforms by inhibiting expression at the nucleic acid level or enzymatic activity at the protein level. The invention allows the identification of and specific inhibition of specific DNA MeTase isoforms involved in tumorigenesis and thus provides a treatment for cancer. The invention further allows identification of and specific inhibition of specific DNA MeTase isoforms involved in cell proliferation and/or differentiation and thus provides a treatment for cell proliferative and/or differentiation disorders.

The inventors have discovered new agents that inhibit specific DNA MeTase isoforms. Accordingly, in a first aspect, the invention provides agents that inhibit one or more specific DNA MeTase isoforms but less than all DNA MeTase isoforms. Such specific DNA MeTase isoforms include without limitation, DNMT-1, DNMT3a and DNMT3b. Non-limiting examples of the new agents include antisense oligonucleotides (oligos) and small molecule inhibitors specific for one or more DNA MeTase isoforms but less than all DNA MeTase isoforms.

The present inventors have surprisingly discovered that specific inhibition of DNMT3a and DNMT3b reverses the tumorigenic state of a transformed cell. The inventors have also surprisingly discovered that the inhibition of the DNMT3a and DNMT3b isoforms dramatically induces growth arrest and apoptosis in cancerous cells. Thus, in certain embodiments of this aspect of the invention, the DNA MeTase isoform that is inhibited is DNMT3a and/or DNMT3b. In certain preferred embodiments, the agent that inhibits the specific DNA MeTase isoform is an oligonucleotide that inhibits expression of a nucleic acid molecule encoding that DNA MeTase isoform. The nucleic acid molecule may be genomic DNA (e.g., a gene), cDNA, or RNA. In some embodiments, the oligonucleotide inhibits transcription of mRNA encoding the DNA MeTase isoform. In other embodiments, the oligonucleotide inhibits translation of the DNA MeTase isoform. In certain embodiments the oligonucleotide causes the degradation of the nucleic acid molecule. Particularly preferred embodiments include antisense oligonucleotides directed to DNMT1, DNMT3a, or DNMT3b. In yet other embodiments of the first aspect, the agent that inhibits a specific DNA MeTase isoform is a small molecule inhibitor that inhibits the activity of one or more specific DNA MeTase isoforms but less than all DNA MeTase isoforms.

In a second aspect, the invention provides a method for inhibiting one or more, but less than all, DNA MeTase isoforms in a cell, comprising contacting the cell with an agent of the first aspect of the invention. In other preferred embodiments, the agent is an antisense oligonucleotide. In certain preferred embodiments, the agent is a small molecule inhibitor. In certain preferred embodiments of the second aspect of the invention, cell proliferation is inhibited in the contacted cell. In preferred embodiments, the cell is a neoplastic cell which may be in an animal, including a human, and which may be in a neoplastic growth. In certain preferred embodiments, the method of the second aspect of the invention further comprises contacting the cell with a DNA MeTase small molecule inhibitor that interacts with and reduces the enzymatic activity of one or more specific DNA MeTase isoforms. In still yet other preferred embodiments of the second aspect of the invention, the method comprises an agent of the first aspect of the invention which is a combination of one or more antisense oligonucleotides and/or one or more small molecule inhibitors of the first aspect of the invention. In certain preferred embodiments, the DNA MeTase isoform is DNMT1, DNMT3a, or DNMT3b. In other certain preferred embodiments, the DNA MeTase isoform is DNMT3a and/or DNMT3b. In some embodiments, the DNA MeTase small molecule inhibitor is operably associated with the antisense oligonucleotide.

In a third aspect, the invention provides a method for inhibiting neoplastic cell proliferation in an animal comprising administering to an animal having at least one neoplastic cell present in its body a therapeutically effective amount of an agent of the first aspect of the invention. In certain preferred embodiments, the agent is an antisense oligonucleotide which is combined with a pharmaceutically acceptable carrier and administered for a therapeutically effective period of time. In certain preferred embodiments, the agent is a small molecule inhibitor which is combined with a pharmaceutically acceptable carrier and administered for a therapeutically effective period of time. In certain preferred embodiments of the this aspect of the invention, cell proliferation is inhibited in the contacted cell. In preferred embodiments, the cell is a neoplastic cell which may be in an animal, including a human, and which may be in a neoplastic growth. In other certain embodiments, the agent is a small molecule inhibitor of the first aspect of the invention which is combined with a pharmaceutically acceptable carrier and administered for a therapeutically effective period of time. In still yet other preferred embodiments of the third aspect of the invention, the method comprises an agent of the first aspect of the invention which is a combination of one or more antisense oligonucleotides and/or one or more small molecule inhibitors of the first aspect of the invention. In certain preferred embodiments, the DNA MeTase isoform is DNMT-1, DNMT3a or DNMT3b. In other certain preferred embodiments, the DNA MeTase isoform is DNMT3a and/or DNMT3b.

In a fourth aspect, the invention provides a method for identifying a specific DNA MeTase isoform that is required for induction of cell proliferation comprising contacting a cell with an agent of the first aspect of the invention. In certain preferred embodiments, the agent is an antisense oligonucleotide that inhibits the expression of a DNA MeTase isoform, wherein the antisense oligonucleotide is specific for a particular DNA MeTase isoform, and thus inhibition of cell proliferation in the contacted cell identifies the DNA MeTase isoform as a DNA MeTase isoform that is required for induction of cell proliferation. In other certain embodiments, the agent is a small molecule inhibitor that inhibits the activity of a DNA MeTase isoform, wherein the small molecule inhibitor is specific for a particular DNA MeTase isoform, and thus inhibition of cell proliferation in the contacted cell identifies the DNA MeTase isoform as a DNA MeTase isoform that is required for induction of cell proliferation. In certain preferred embodiments, the cell is a neoplastic cell, and the induction of cell proliferation is tumorigenesis. In still yet other preferred embodiments of the fourth aspect of the invention, the method comprises an agent of the first aspect of the invention which is a combination of one or more antisense oligonucleotides and/or one or more small molecule inhibitors of the first aspect of the invention. In certain preferred embodiments, the DNA MeTase isoform is DNMT-1, DNMT3a or DNMT3b. In other certain preferred embodiments, the DNA MeTase isoform is DNMT3a and/or DNMT3b.

In a fifth aspect, the invention provides a method for identifying a DNA MeTase isoform that is involved in induction of cell differentiation, comprising contacting a cell with an agent that inhibits the expression of a DNA MeTase isoform, wherein induction of differentiation in the contacted cell identifies the DNA MeTase isoform as a DNA MeTase isoform that is involved in induction of cell differentiation. In certain preferred embodiments, the agent is an antisense oligonucleotide of the first aspect of the invention. In other certain preferred embodiments, the agent is a small molecule inhibitor of the first aspect of the invention. In still other certain embodiments, the cell is a neoplastic cell. In still yet other preferred embodiments of the fifth aspect of the invention, the method comprises an agent of the first aspect of the invention which is a combination of one or more antisense oligonucleotides and/or one or more small molecule inhibitors of the first aspect of the invention. In certain preferred embodiments, the DNA MeTase isoform is DNMT-1, DNMT3a or DNMT3b. In other certain preferred embodiments, the DNA MeTase isoform is DNMT3a and/or DNMT3b. In a sixth aspect, the invention provides a method for inhibiting neoplastic cell growth in an animal comprising administering to an animal having at least one neoplastic cell present in its body a therapeutically effective amount of an agent of the first aspect of the invention. In certain embodiments thereof, the agent is an antisense oligonucleotide, which is combined with a pharmaceutically acceptable carrier and administered for a therapeutically effective period of time.

In a seventh aspect, the invention provides a method for identifying a DNA MeTase isoform that is involved in induction of cell differentiation, comprising contacting a cell with an antisense oligonucleotide that inhibits the expression of a DNA MeTase isoform, wherein induction of differentiation in the contacted cell identifies the DNA MeTase isoform as a DNA MeTase isoform that is involved in induction of cell differentiation. Preferably, the cell is a neoplastic cell. In certain preferred embodiments, the DNA MeTase isoform is DNMT-1, DNMT3a or DNMT3b. In other certain preferred embodiments, the DNA MeTase isoform is DNMT3a and/or DNMT3b.

In an eighth aspect, the invention provides a method for inhibiting cell proliferation in a cell comprising contacting a cell with at least two agents selected from the group consisting of an antisense oligonucleotide from the first aspect of the invention that inhibits expression of a specific DNA MeTase isoform, a small molecule inhibitor from the first aspect of the invention that inhibits a specific DNA MeTase isoform, an antisense oligonucleotide that inhibits a DNA methyltransferase, and a small molecule that inhibits a DNA methyltransferase. In one embodiment, the inhibition of cell growth of the contacted cell is greater than the inhibition of cell growth of a cell contacted with only one of the agents. In certain embodiments, each of the agents selected from the group is substantially pure. In preferred embodiments, the cell is a neoplastic cell. In yet additional preferred embodiments, the agents selected from the group are operably associated. In certain preferred embodiments, the DNA MeTase isoform is DNMT-1, DNMT3a or DNMT3b. In other certain preferred embodiments, the DNA MeTase isoform is DNMT3a and/or DNMT3b.

In a ninth aspect, the invention provides a method for modulating cell proliferation or differentiation, comprising contacting a cell with an agent of the first aspect of the invention, wherein one or more, but less than all, DNA MeTase isoforms are inhibited, which results in a modulation of proliferation or differentiation. In certain embodiments, the agent is an antisense oligonucleotide of the first aspect of the invention. In other certain preferred embodiments, the agent is a small molecule inhibitor of the first aspect of the invention. In preferred embodiments, the cell proliferation is neoplasia. In still yet other preferred embodiments of the this aspect of the invention, the method comprises an agent of the first aspect of the invention which is a combination of one or more antisense oligonucleotides and/or one or more small molecule inhibitors of the first aspect of the invention. In certain preferred embodiments, the DNA MeTase isoform is DNMT-1, DNMT3a or DNMT3b. In other certain preferred embodiments, the DNA MeTase isoform is DNMT3a and/or DNMT3b.

In an tenth aspect, the invention provides a method for inhibiting cell proliferation in a cell comprising contacting a cell with at least two agents selected from the group consisting of an antisense oligonucleotide from the first aspect of the invention that inhibits expression of a specific DNA MeTase isoform, a small molecule inhibitor that inhibits a specific DNA MeTase isoform, an antisense oligonucleotide that inhibits a histone deactylase, and a small molecule that inhibits a histone deactylase. In one embodiment, the inhibition of cell growth of the contacted cell is greater than the inhibition of cell growth of a cell contacted with only one of the agents. In certain embodiments, each of the agents selected from the group is substantially pure. In preferred embodiments, the cell is a neoplastic cell. In yet additional preferred embodiments, the agents selected from the group are operably associated.

In an eleventh aspect, the invention provides a method for inhibiting cell proliferation in a cell comprising contacting a cell with at least two agents selected from the group consisting of an antisense oligonucleotide from the first aspect of the invention that inhibits expression of a specific DNA MeTase isoform, a small molecule inhibitor that inhibits a specific DNA MeTase isoform, an antisense oligonucleotide that inhibits a histone deactylase, and a small molecule that inhibits a histone deactylase. In one embodiment, the inhibition of cell growth of the contacted cell is greater than the inhibition of cell growth of a cell contacted with only one of the agents. In certain embodiments, each of the agents selected from the group is substantially pure. In preferred embodiments, the cell is a neoplastic cell. In yet additional preferred embodiments, the agents selected from the group are operably associated.

### BRIEF DESCRIPTION OF THE DRAWINGS

FIG. 1A is a schematic diagram providing the structures and Genbank accession numbers of the DNA methyltransferase genes, DNMT1, DNMT3a and DNMT3b.

FIG. 1B is a schematic diagram providing the nucleotide sequence of DNMT1 cDNA, as provided in GenBank Accession No.(NM_{—}001379).

FIG. 1C is a schematic diagram providing the nucleotide sequence of DNMT3a cDNA, as provided in GenBank Accession No.(AF_{—}067972).

FIG. 1D is a schematic diagram providing the nucleotide sequence of DNMT3b, as provided in GenBank Accession No. (NM_{—}006892).

FIG. 1E is a schematic diagram providing the nucleotide sequence of DNMT3b3, as provided in GenBank Accession No. (AF_{—}156487).

FIG. 1F is a schematic diagram providing the nucleotide sequence of DNMT3b4, as provided in GenBank Accession No. (AF_{—}129268).

FIG. 1G is a schematic diagram providing the nucleotide sequence of DNMT3b, as provided in GenBank Accession No. (AF_{—}129269).

FIG. 2 is a schematic diagram providing the structure of the DNMT3a cDNA and the position of antisense oligonucleotides tested in initial screens. Numbers in parenthesis indicate the starting position of the antisense oligonucleotides on the DNMT3a sequence. The sequence and position of the most active antisense inhibitors identified from the screen is also shown.

FIG. 3 is a schematic diagram providing the structure of the DNMT3b cDNA and the position of antisense oligonucleotides tested in initial screens. Numbers in parenthesis indicate the starting position of the antisense oligonucleotides on the DNMT3b sequence. The sequence and position of the most active antisense inhibitors identified from the screen is also shown.

FIG. 4 is a representation of a Northern blot demonstrating the dose dependent effect of DNMT3a antisense oligonucleotide (SEQ ID NO: 33) on the expression of DNMT3a mRNA in A549 human non small cell lung cancer cells. Also demonstrated is the specificity of SEQ ID NO: 33 for DNMT3a as non target mRNAs DNMT1, DNMT3b and Glyceraldehyde 3′-phosphate Dehydrogenase are not effected.

FIG. 5 is a representation of a Northern blot demonstrating the dose dependent effect of DNMT3b antisense oligonucleotide (SEQ ID NO: 18) on the expression of DNMT3b mRNA in A549 human non small cell lung cancer cells. Also demonstrated is the specificity of SEQ ID NO: 18 for DNMT3a as non target mRNAs DNMT1, DNMT3a and Glyceraldehyde 3′-phosphate Dehydrogenase are not effected.

FIG. 6 is a representation of a Western blot demonstrating the dose dependent effect of DNMT3b antisense inhibitor SEQ ID NO: 18 on the level of DNMT3b protein in T24 human bladder cancer cells and A549 human non small cell lung cancer cells. Cells were treated for 48 hrs with increasing doses of SEQ ID NO: 18 after which cells were harvested and DNMT3b levels were determined by Western blot with a DNMT3b specific antibody.

FIG. 7 is a graphic representation demonstrating the apoptotic effect of Dnt3a and DNMT3b inhibition on A549 human non small cell lung cancer cells.

FIG. 8 is a graphic representation demonstrating the Dose dependent apoptotic effect of Dnt3b inhibition on A549 human non small cell lung cancer cells by three DNMT3b antisense inhibitors.

FIG. 9 is a graphic representation demonstrating the Dose dependent apoptotic effect of Dnt3b inhibition on T24 human non small cell lung cancer cells by three DNMT3b antisense inhibitors.

FIG. 10 is a graphic representation demonstrating the cancer specific apoptotic effect of DNMT3b inhibition. DNMT3b inhibitor SEQ ID NO: 18 induced apotosis in A549 cells yet similar treatment of the two normal cell lines HMEC and MRHF produced no apoptosis.

FIG. 11A is a graphic representation demonstrating the dose dependent effect of Dnmt3b AS1 antisense oligonucleotides on the proliferation of human A549 cancer cells.

FIG. 11B is a graphic representation demonstrating the cancer specificity of antiproliferative effect of Dnmt3a and Dnmt3b inhibition. Inhibition of Dnmt3a or Dnmt3b produces antiproliferative effects of cancer cells but not affect the proliferation of the human normal skin fibroblast cell line MRHF.

### DETAILED DESCRIPTION OF THE PREFERRED EMBODIMENTS

The invention provides methods and agents for inhibiting specific DNA MeTase isoforms by inhibiting expression at the nucleic acid level or protein activity at the enzymatic level. The invention allows the identification of and specific inhibition of specific DNA MeTase isoforms involved in tumorigenesis and thus provides a treatment for cancer. The invention further allows identification of and specific inhibition of specific DNA MeTase isoforms involved in cell proliferation and/or differentiation and thus provides a treatment for cell proliferative and/or differentiation disorders.

The patent and scientific literature referred to herein establishes knowledge that is available to those with skill in the art. The issued patents, applications, and references, including GenBank database sequences, that are cited herein are hereby incorporated by reference to the same extent as if each was specifically and individually indicated to be incorporated by reference.

In a first aspect, the invention provides agents that inhibit one or more DNA MeTase isoforms, but less than all specific DNA MeTase isoforms. As used herein interchangeably, the terms “DNA MeTase”, “DNMT”, “DNA MeTase isoform”, “DNMT isoform” and similar terms are intended to refer to any one of a family of enzymes that add a methyl groups to the C5 position of cytosine in DNA. Preferred DNA MeTase isoforms include maintenance and de novo methyltransferases. Specific DNA MeTases include without limitation, DNMT-1, DNMT3a, and DNMT3b. By way of non-limiting example, useful agents that inhibit one or more DNA MeTase isoforms, but less than all specific DNA MeTase isoforms, include antisense oligonucleotides and small molecule inhibitors.

The present inventors have surprisingly discovered that specific inhibition of DNMT-1 reverses the tumorigenic state of a transformed cell. The inventors have also surprisingly discovered that the inhibition of the DNMT3b and/or DNMT3b isoform dramatically induces growth arrest and apoptosis in cancerous cells. Thus, in certain embodiments of this aspect of the invention, the DNA MeTase isoform that is inhibited is DNMT3a and/or DNMT3b.

Preferred agents that inhibit DNMT3a and/or DNMT3b dramatically inhibit growth of human cancer cells, independent of p53 status. These agents significantly induce apoptosis in the cancer cells and cause dramatic growth arrest. Inhibitory agents that achieve one or more of these results are considered within the scope of this aspect of the invention. By way of non-limiting example, antisense oligonucleotides and/or small molecule inhibitors of DNMT3a and/or DNMT3b are useful for the invention.

In certain preferred embodiments, the agent that inhibits the specific DNMT isoform is an oligonucleotide that inhibits expression of a nucleic acid molecule encoding a specific DNA MeTase isoform. The nucleic acid molecule may be genomic DNA (e.g., a gene), cDNA, or RNA. In other embodiments, the oligonucleotide ultimately inhibits translation of the DNA MeTase. In certain embodiments the oligonucleotide causes the degradation of the nucleic acid molecule. Preferred antisense oligonucleotides have potent and specific antisense activity at nanomolar concentrations.

The antisense oligonucleotides according to the invention are complementary to a region of RNA or double-stranded DNA that encodes a portion of one or more DNA MeTase isoforms (taking into account that homology between different isoforms may allow a single antisense oligonucleotide to be complementary to a portion of more than one isoform).

For purposes of the invention, the term “complementary” means having the ability to hybridize to a genomic region, a gene, or an RNA transcript thereof under physiological conditions. Such hybridization is ordinarily the result of base-specific hydrogen bonding between complementary strands, preferably to form Watson-Crick or Hoogsteen base pairs, although other modes of hydrogen bonding, as well as base stacking can lead to hybridization. As a practical matter, such hybridization can be inferred from the observation of specific gene expression inhibition, which may be at the level of transcription or translation (or both).

For purposes of the invention, the term “oligonucleotide” includes polymers of two or more deoxyribonucleosides, ribonucleosides, or 2′-O-substituted ribonucleoside residues, or any combination thereof. Preferably, such oligonucleotides have from about 8 to about 50 nucleoside residues, and most preferably from about 12 to about 30 nucleoside residues. The nucleoside residues may be coupled to each other by any of the numerous known internucleoside linkages. Such internucleoside linkages include without limitation phosphorothioate, phosphorodithioate, alkylphosphonate, alkylphosphonothioate, phosphotriester, phosphoramidate, siloxane, carbonate, carboxymethylester, acetamidate, carbamate, thioether, bridged phosphoramidate, bridged methylene phosphonate, bridged phosphorothioate, and sulfone internucleotide linkages. In certain preferred embodiments, these internucleoside linkages may be phosphodiester, phosphotriester, phosphorothioate, or phosphoramidate linkages, or combinations thereof. The term oligonucleotide also encompasses such polymers having chemically modified bases or sugars and/or having additional substituents, including without limitation lipophilic groups, intercalating agents, diamines, and adamantane. The term oligonucleotide also encompasses such polymers as PNA and LNA. For purposes of the invention the term “2′-O-substituted” means substitution of the 2′ position of the pentose moiety with an -O-lower alkyl group containing 1-6 saturated or unsaturated carbon atoms, or with an -O-aryl or allyl group having 2-6 carbon atoms, wherein such alkyl, aryl, or allyl group may be unsubstituted or may be substituted, e.g., with halo, hydroxy, trifluoromethyl, cyano, nitro, acyl, acyloxy, alkoxy, carboxyl, carbalkoxyl, or amino groups; or such 2′ substitution may be with a hydroxy group (to produce a ribonucleoside), an amino or a halo group, but not with a 2′-H group.

Particularly preferred antisense oligonucleotides utilized in this aspect of the invention include chimeric oligonucleotides and hybrid oligonucleotides.

For purposes of the invention, a “chimeric oligonucleotide” refers to an oligonucleotide having more than one type of internucleoside linkage. One preferred embodiment of such a chimeric oligonucleotide is a chimeric oligonucleotide comprising a phosphorothioate, phosphodiester or phosphorodithioate region, preferably comprising from about 2 to about 12 nucleotides, and an alkylphosphonate or alkylphosphonothioate region (see e.g., Pederson et al. U.S. Pat. Nos. 5,635,377 and 5,366,878). Preferably, such chimeric oligonucleotides contain at least three consecutive internucleoside linkages selected from phosphodiester and phosphorothioate linkages, or combinations thereof.

For purposes of the invention, a “hybrid oligonucleotide” refers to an oligonucleotide having more than one type of nucleoside. One preferred embodiment of such a hybrid oligonucleotide comprises a ribonucleotide or 2′-O-substituted ribonucleotide region, preferably comprising from about 2 to about 12 2′-O-substituted nucleotides, and a deoxyribonucleotide region. Preferably, such a hybrid oligonucleotide will contain at least three consecutive deoxyribonucleosides and will also contain ribonucleosides, 2′-O-substituted ribonucleosides, or combinations thereof (see e.g., Metelev and Agrawal, U.S. Pat. Nos. 5,652,355 and 5,652,356).

The exact nucleotide sequence and chemical structure of an antisense oligonucleotide utilized in the invention can be varied, so long as the oligonucleotide retains its ability to inhibit expression of a specific DNA MeTase isoform or inhibit one or more DNA MeTase isoforms, but less than all specific DNA MeTase isoforms. This is readily determined by testing whether the particular antisense oligonucleotide is active by quantitating the amount of mRNA encoding a specific DNA MeTase isoform, quantitating the amount of DNA MeTase isoform protein, quantitating the DNA MeTase isoform enzymatic activity, or quantitating the ability of the DNA MeTase isoform to inhibit cell growth in a an in vitro or in vivo cell growth assay, all of which are described in detail in this specification. The term “inhibit expression” and similar terms used herein are intended to encompass any one or more of these parameters.

Antisense oligonucleotides utilized in the invention may conveniently be synthesized on a suitable solid support using well-known chemical approaches, including H-phosphonate chemistry, phosphoramidite chemistry, or a combination of H-phosphonate chemistry and phosphoramidite chemistry (i.e., H-phosphonate chemistry for some cycles and phosphoramidite chemistry for other cycles). Suitable solid supports include any of the standard solid supports used for solid phase oligonucleotide synthesis, such as controlled-pore glass (CPG) (see, e.g., Pon, R. T., Methods in Molec. Biol. 20: 465-496, 1993).

Antisense oligonucleotides according to the invention are useful for a variety of purposes. For example, they can be used as “probes” of the physiological function of specific DNA MeTase isoforms by being used to inhibit the activity of specific DNA MeTase isoforms in an experimental cell culture or animal system and to evaluate the effect of inhibiting such specific DNA MeTase isoform activity. This is accomplished by administering to a cell or an animal an antisense oligonucleotide that inhibits the expression of one or more DNA MeTase isoforms according to the invention and observing any phenotypic effects. In this use, the antisense oligonucleotides according to the invention is preferable to traditional “gene knockout” approaches because it is easier to use, and can be used to inhibit specific DNA MeTase isoform activity at selected stages of development or differentiation.

Preferred antisense oligonucleotides of the invention inhibit either the transcription of a nucleic acid molecule encoding the DNA MeTase isoform, and/or the translation of a nucleic acid molecule encoding the DNA MeTase isoform, and/or lead to the degradation of such nucleic acid. DNA MeTase-encoding nucleic acids may be RNA or double stranded DNA regions and include, without limitation, intronic sequences, untranslated 5′ and 3′ regions, intron-exon boundaries as well as coding sequences from a DNA MeTase family member gene. (See, e.g., Yoder, J. A., et al. (1996) J. Biol. Chem. 271:31092-31097; Xie, S., et al. (1999) Gene 236:87-95; and Robertson, K. D., et al. (1999) Nucleic Acids Research 27:2291-2298).

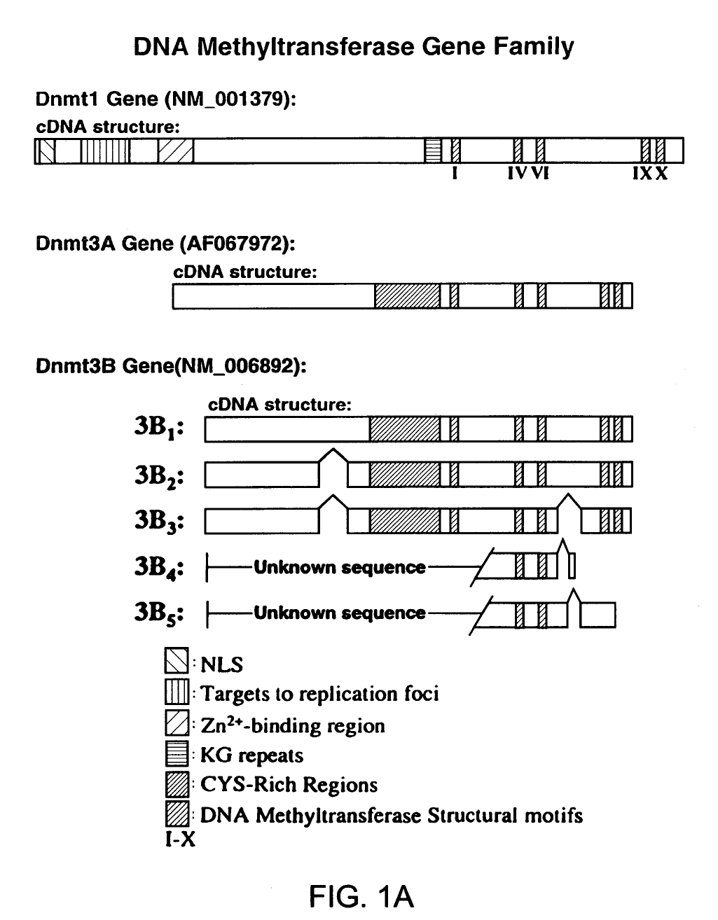

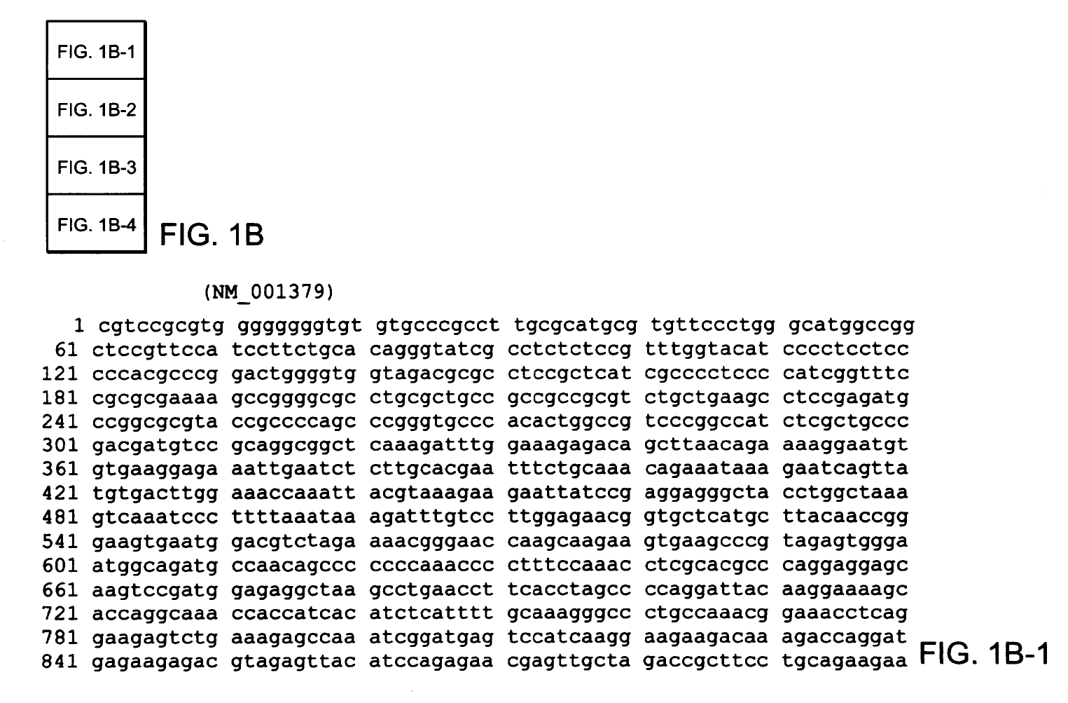

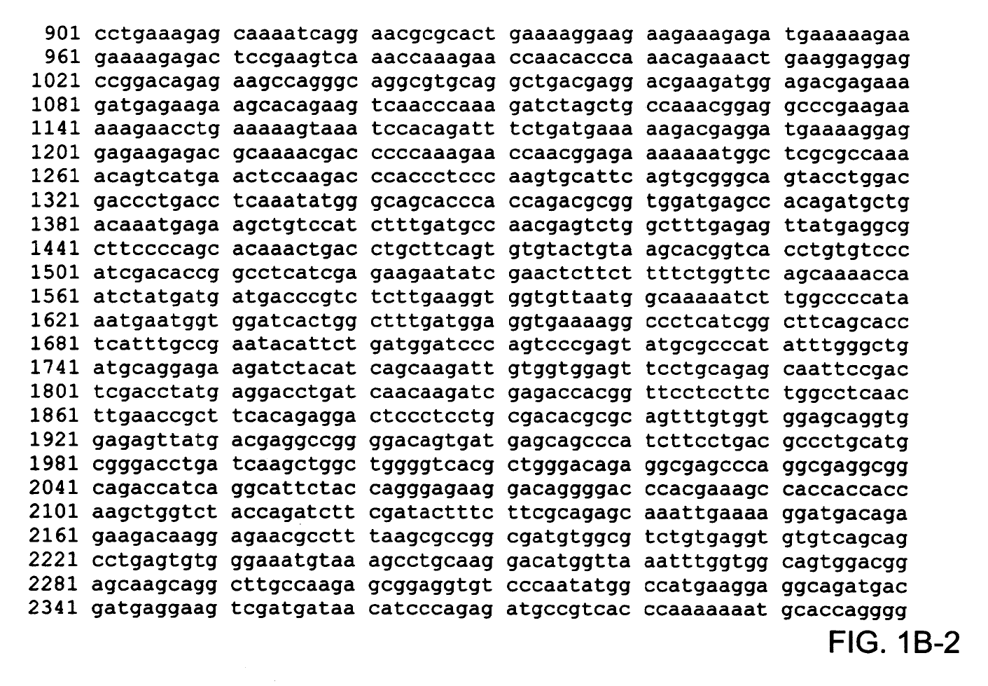

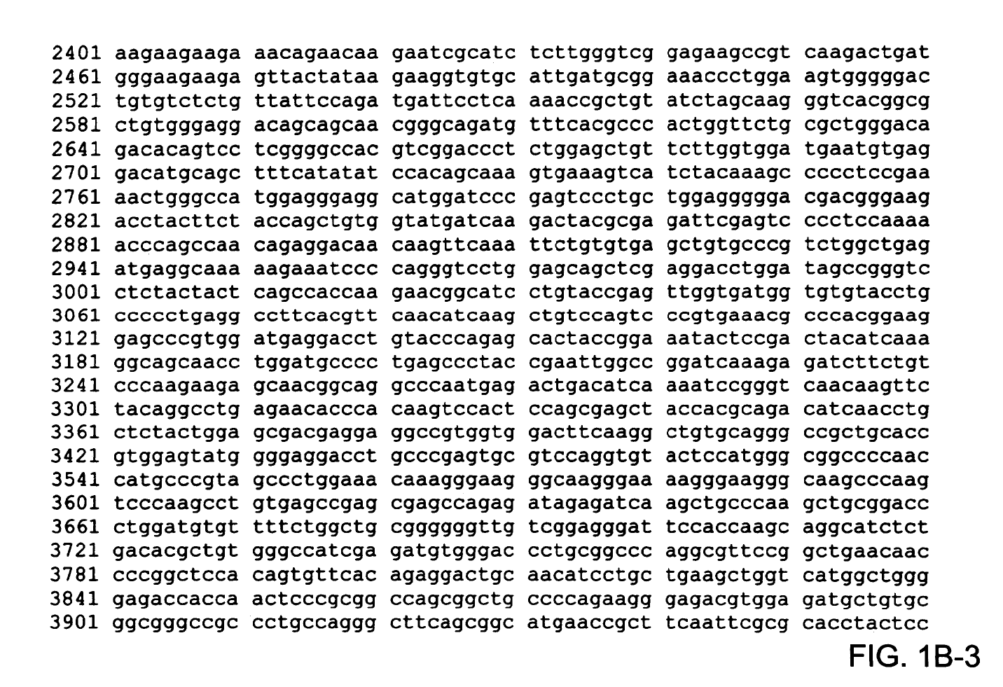

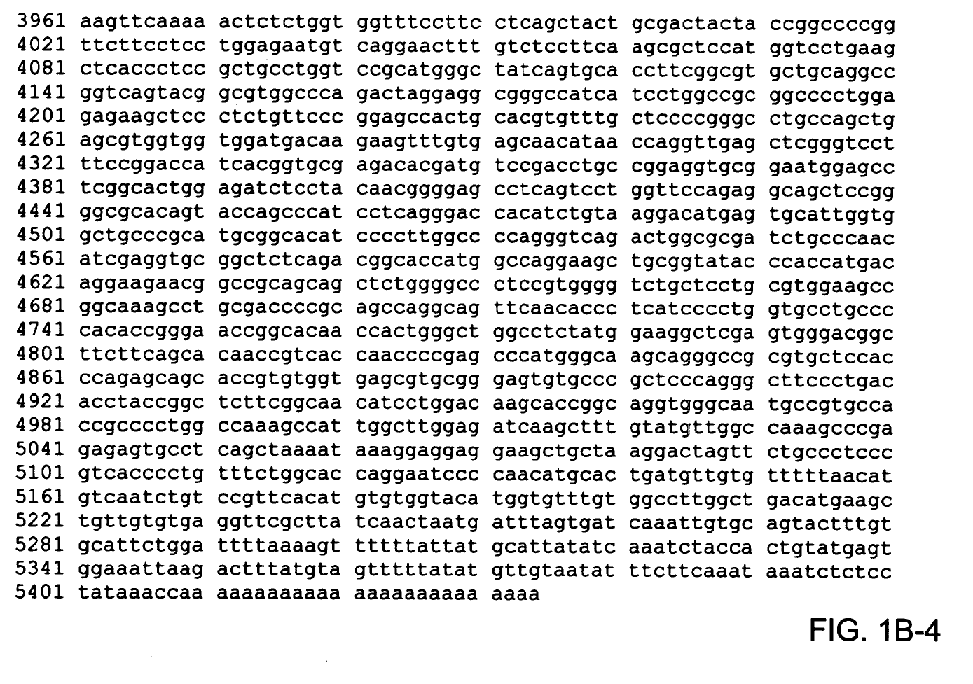

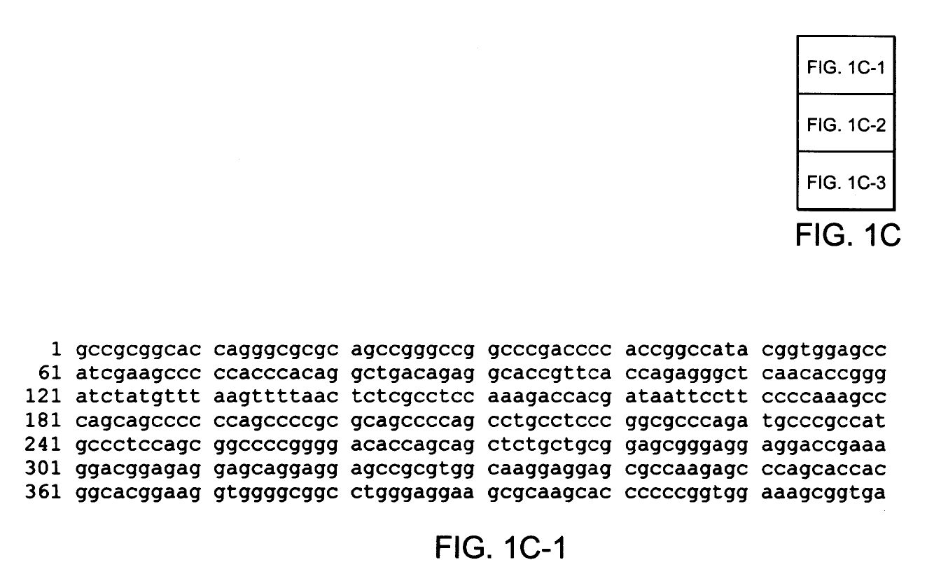

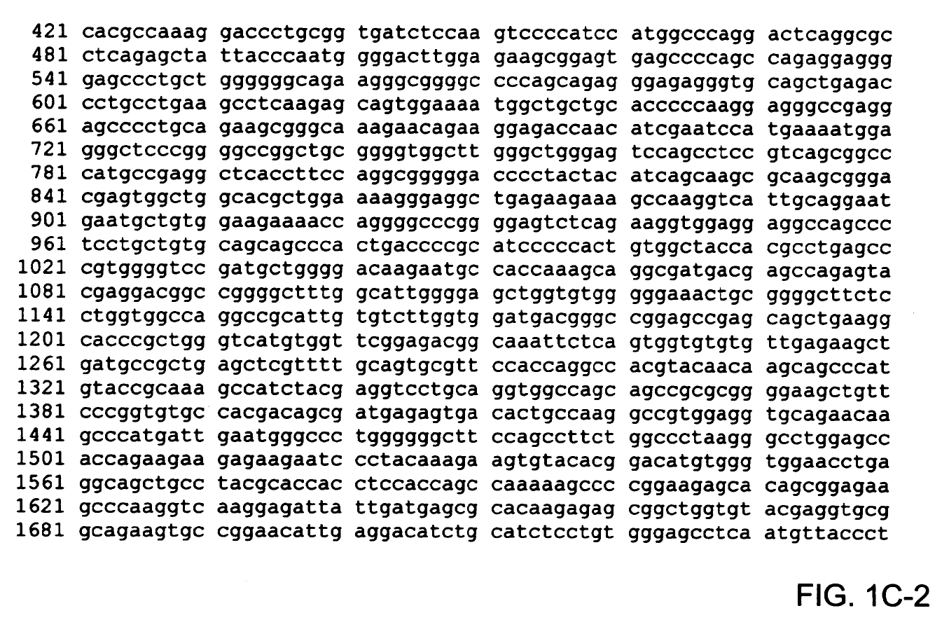

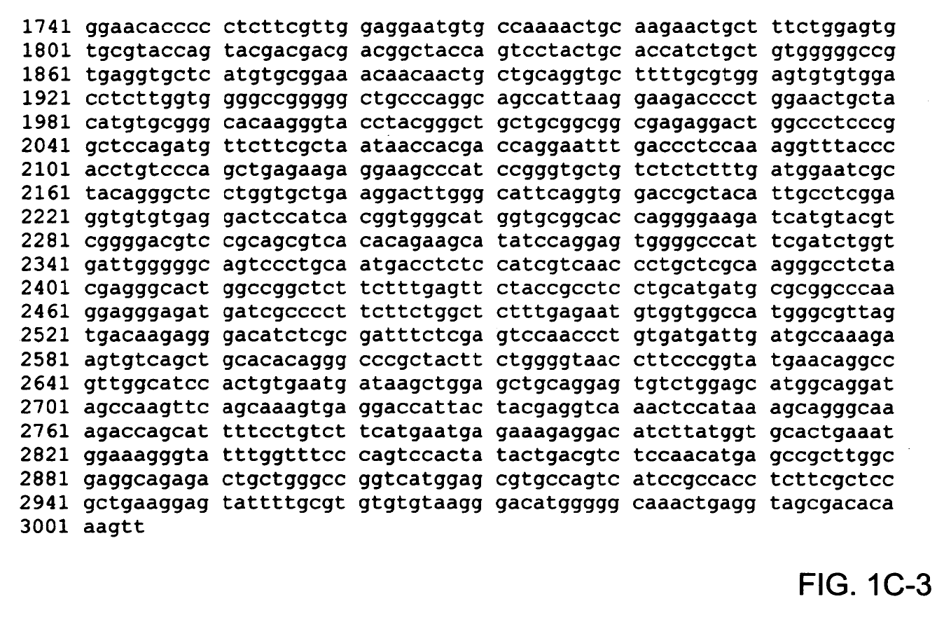

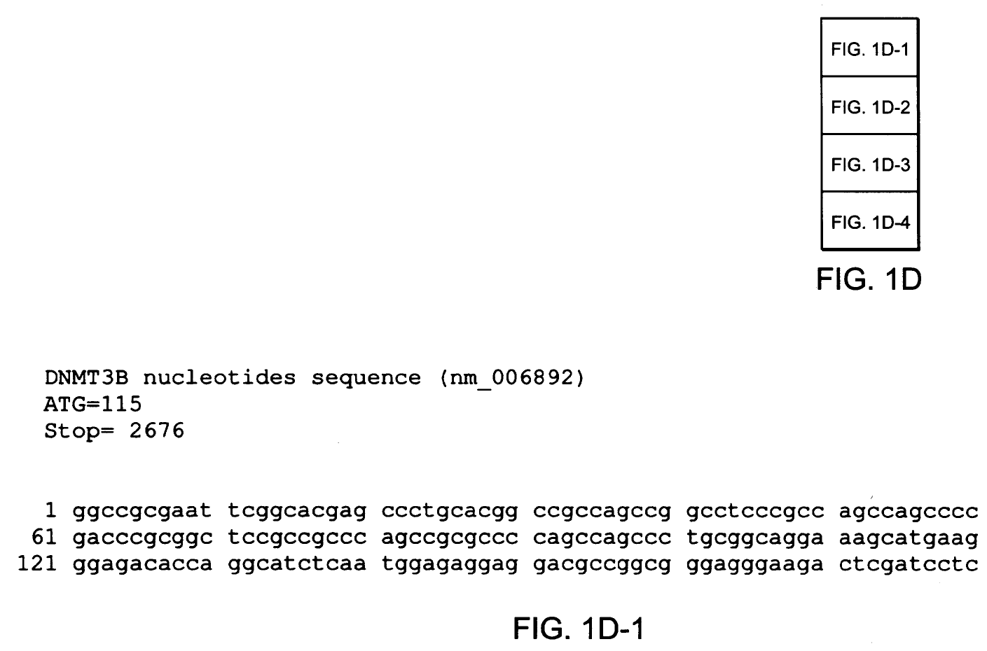

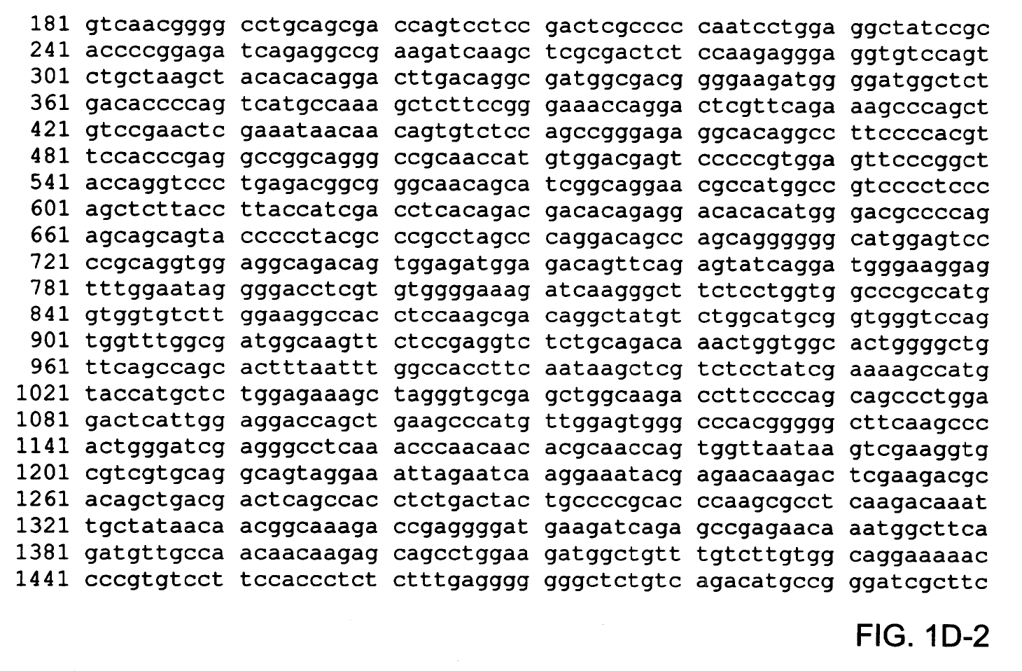

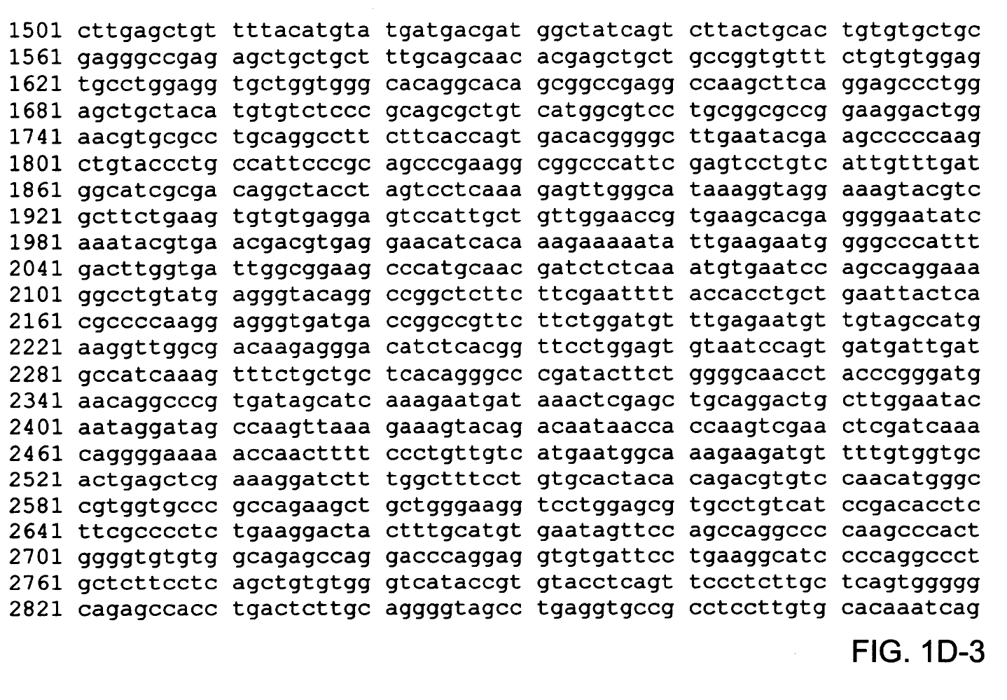

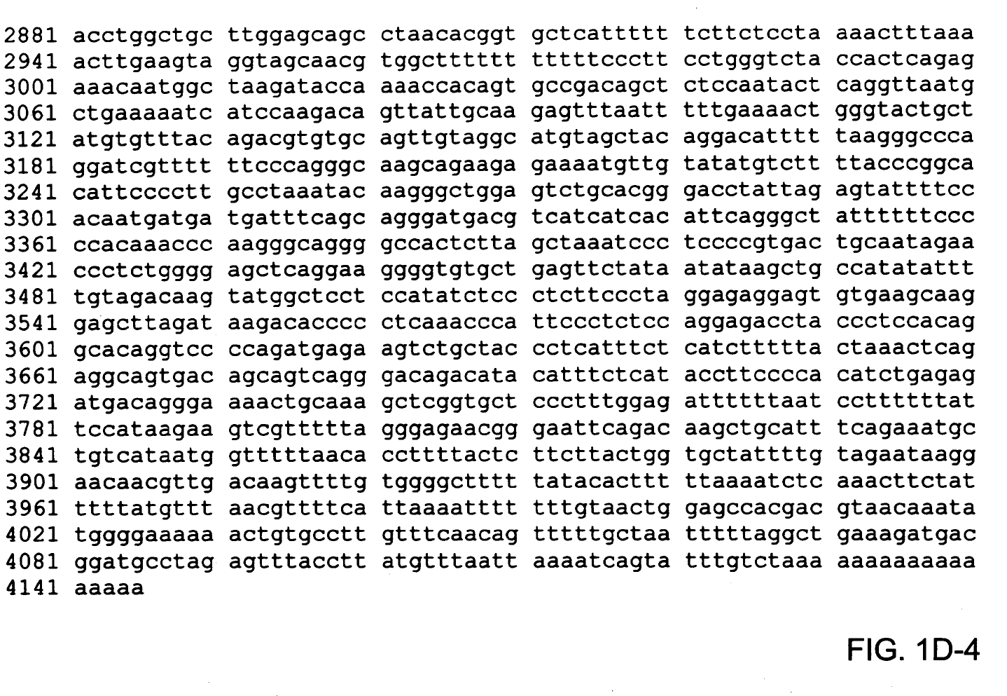

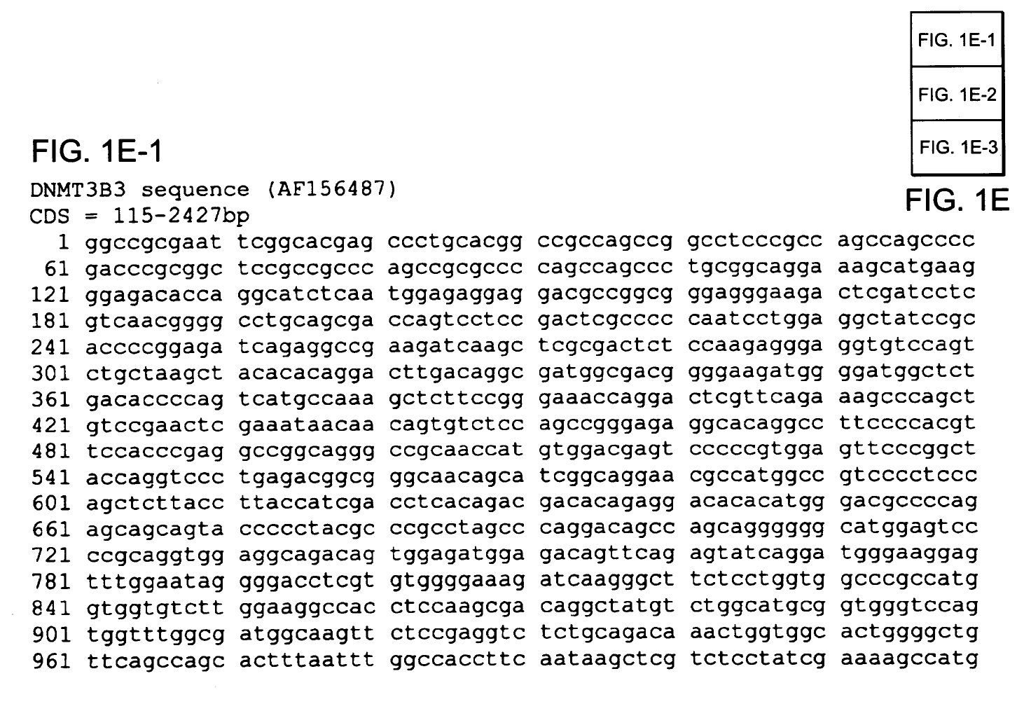

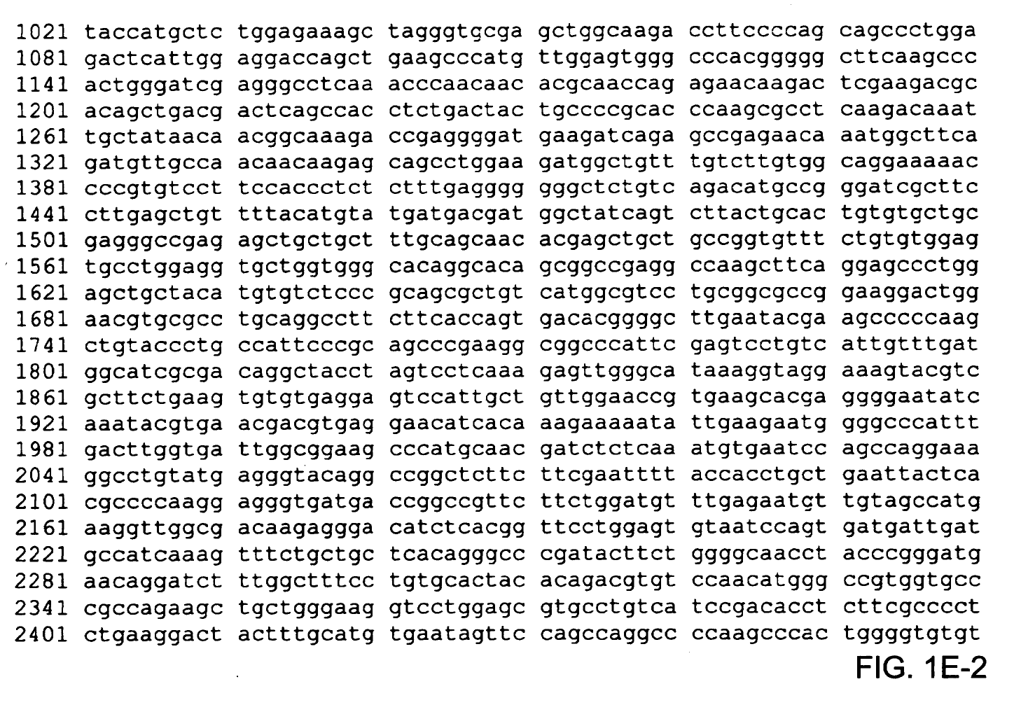

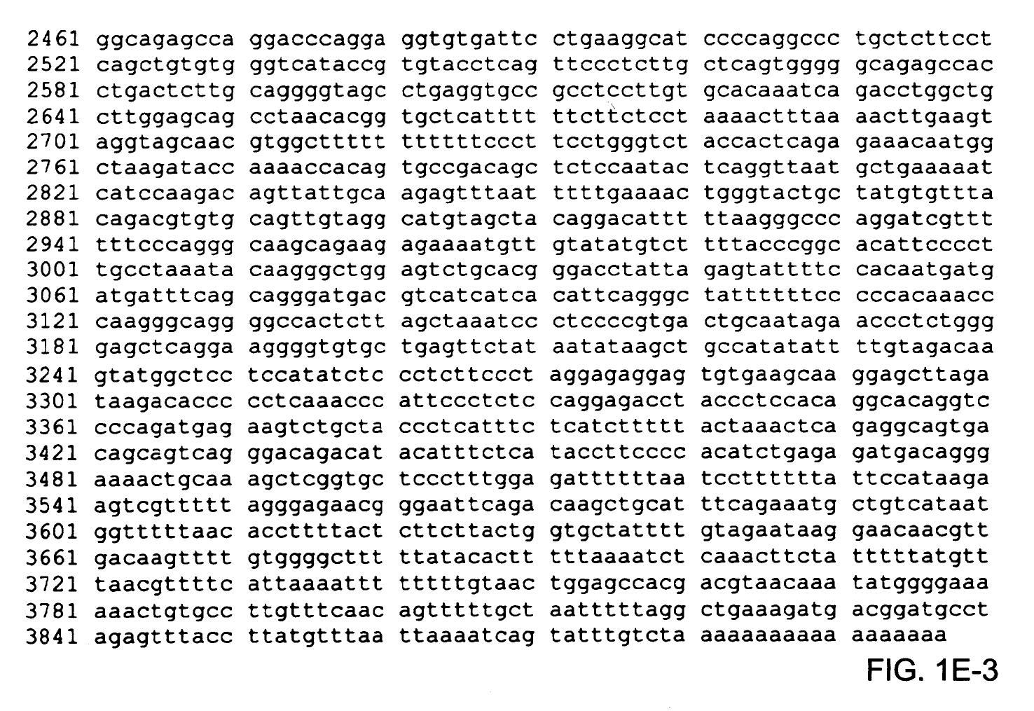

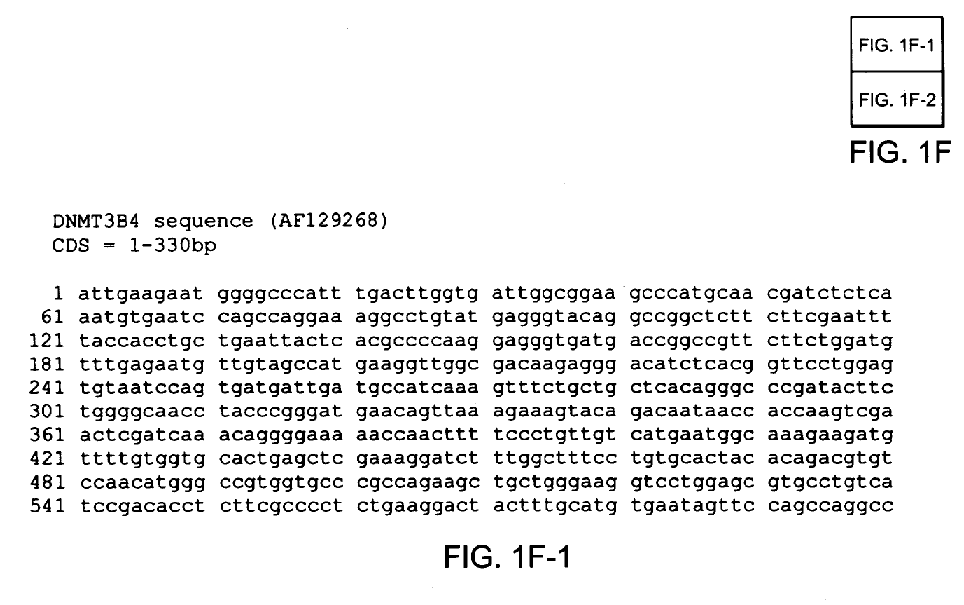

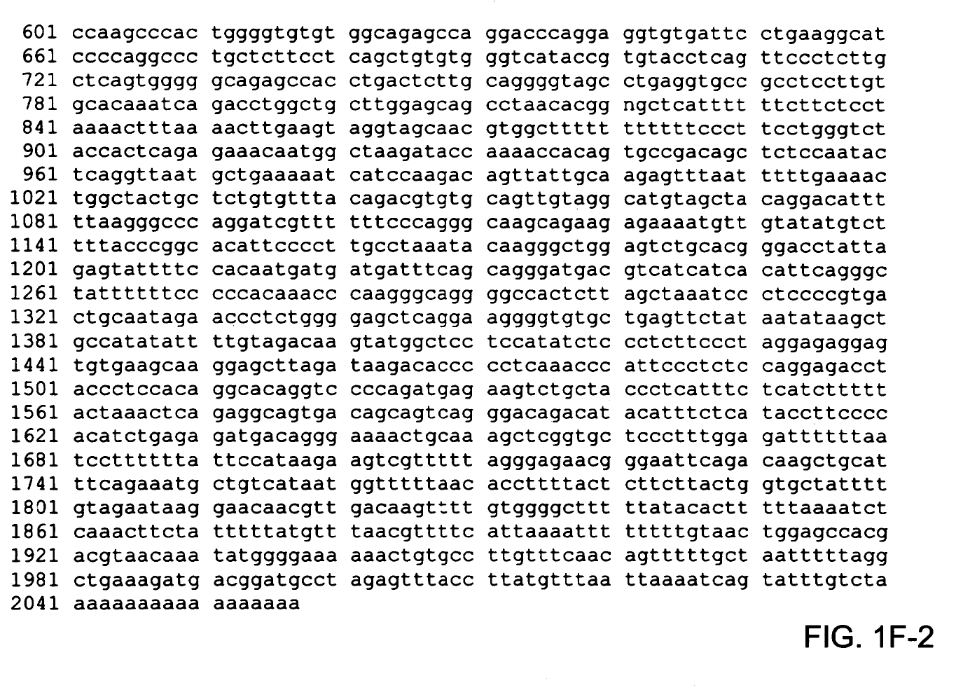

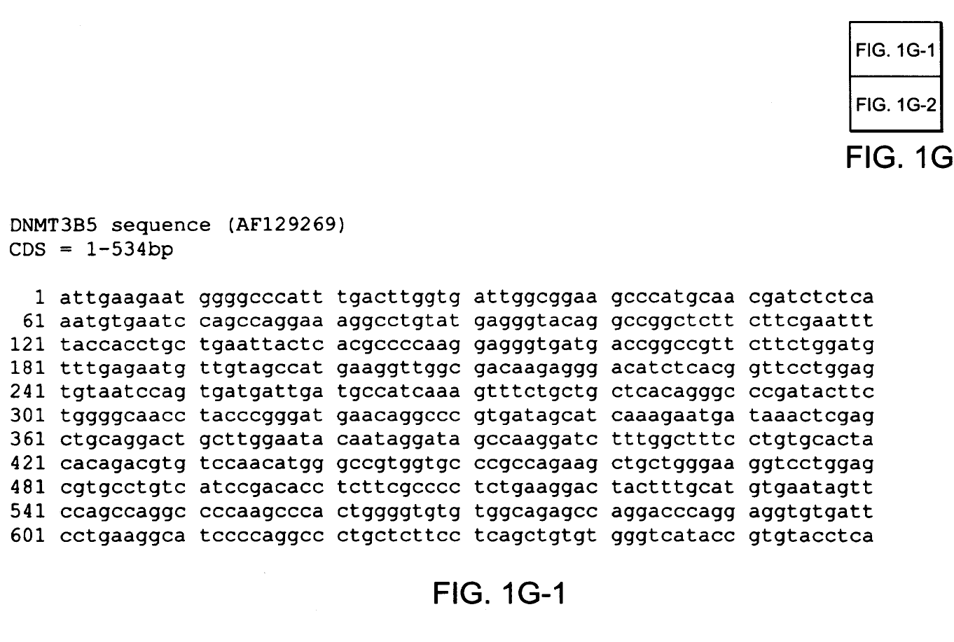

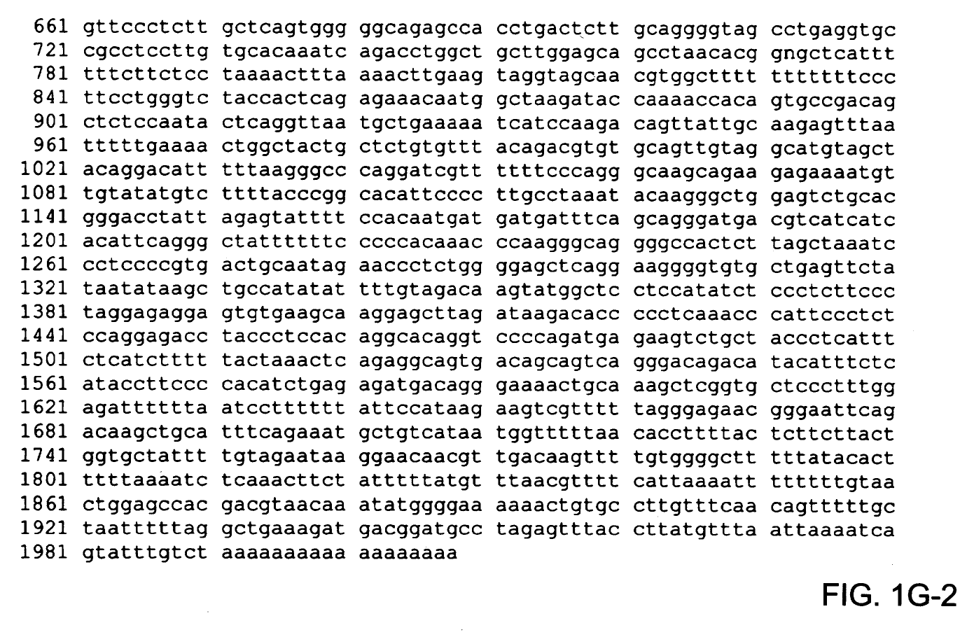

Particularly preferred non-limiting examples of antisense oligonucleotides of the invention are complementary to regions of RNA or double-stranded DNA encoding a DNA MeTase isoform (e.g., DNMT-1, DNMT3a, DNMT3b (also known as DNMT3b1), DNMT3b2, DNMT3b3, DNMT3b3, DNMT3b4, DNMT3b5). (see e.g., GenBank Accession No. NM_{—}001379 for human DNMT-1 (FIG. 1B); GenBank Accession No. AF_{—}067972 for human DNMT3a, (FIG. 1C); GenBank Accession Nos. NM_{—}006892, AF_{—}156488, AF_{—}176228, and XM_{—}009449 for human DNMT3b (FIG. 1D); nucleotide positions 115-1181 and 1240-2676 of GenBank No. NM_{—}006892 for human DNMT3b2, GenBank Accession No. AF_{—}156487 for human DNMT3b3 (FIG. 1E), GenBank Accession No. AF_{—}129268 for human DNMT3b4 (FIG. 1F), and GenBank Accession No. AF_{—}129269 for human DNMT3b5 (FIG. 1G).

As used herein, a reference to any one of the specific DNA MeTases isoforms includes reference to all RNA splice variants of that particular isoform. By way of non-limiting example, reference to DNMT3b is meant to include the splice variants DNMTb2, DNMTb3, DNMTb4, and DNMTb5.

The sequences encoding DNA MeTases from non-human animal species are also known (see, for example, GenBank Accession Numbers AF_{—}175432 (murine DNMT-1); NM_{—}010068 (murine DNMT3a); and NM_{—}007872 (murine DNMT3b). Accordingly, the antisense oligonucleotides of the invention may also be complementary to regions of RNA or double-stranded DNA that encode DNA MeTases from non-human animals. Antisense oligonucleotides according to these embodiments are useful as tools in animal models for studying the role of specific DNA MeTase isoforms.

Particularly, preferred oligonucleotides have nucleotide sequences of from about 13 to about 35 nucleotides which include from about 13 to all of a nucleotide sequence shown in Table 1 and Table 2. Yet additional particularly preferred oligonucleotides have nucleotide sequences of from about 15 to about 26 nucleotides. Most preferably, the oligonucleotides shown below have phosphorothioate backbones, are 20-26 nucleotides in length, and are modified such that the terminal four nucleotides at the 5′ end of the oligonucleotide and the terminal four nucleotides at the 3′ end of the oligonucleotide each have 2′-O-methyl groups attached to their sugar residues.

Antisense oligonucleotides used in the present study are shown in Table 1 and Table 2.

TABLE 1
Sequences of Human DNA MeTase DNMT1 Antisense
(AS) Oligonucleotides and Their Mismatch (MM)
Oligonucleotides

| | | | | |
|---|---|---|---|---|
| | (SEQ | | (SEQ | |
| | ID | IC₅₀ | ID | IC₅₀ |
| Sequence | NO) | (nM)¹ | NO) | (nM)² |

| | | | | |
|---|---|---|---|---|
| 5′CAGGTAGCCCTCCTCGGAT 03′ | [4] | 90 | [11] | 70 |
| 5′AAGCATGAGCACCGTTCTCC 3′ | [5] | 66 | [12] | 43 |
| 5′TTCATGTCAGCCAAGGCCAC 3′ | [6] | 67 | [13] | 60 |
| 5′CGAACCTCACACAACAGCTT 3′ | [7] | 96 | [14] | 75 |
| 5′GATAAGCGAACCTCACACAA 3′ | [8] | 90 | [15] | 81 |
| 5′CCAAGGCCACAAACACCATG 3′ | [9] | 66 | [16] | 60 |
| 5′CATCTGCCATTCCCACTCTA 3′ | [10]³ | 133 | [17] | 114 |
| Scrambled sequence | -- | >>250 | -- | >>250 |

¹oligodeoxynucleoside phosphorothioate
²hybrid oligonucleoside phosphorothioate with four 2-o-methyl ribonucleosides at each end and deoxyribonucleosides in the middle, any thymidine within four nucleotides from either the 5′ or the 3′ end of the antisense oligonucleotide is substituted with a uridine in the hybrid oligonucleotides.
³control prior art oligonucleotide spanning translation initiation site

TABLE 2
Sequences of Human DNA MeTase DNMT3a and DNMT3b Antisense (AS)
Oligonucleotides and Their Mismatch (MM) Oligonucleotides

| | | | | |
|---|---|---|---|---|
| | | Nucleotide | | |
| Target | Accession Number | Position | Chemistry | Sequence |

| | | | | | |
|---|---|---|---|---|---|
| DNMT3B AS | NM_006892 | 3′UTR (3993) | PTI | 5′cgtcgtggctccagttacaa3′ | (SEQ ID NO:18) |
| DNMT3B MM | NM_006892 | | PTI | 5′cctcgtcggtcgacttagaa3′ | (SEQ ID NO:19) |
| DNMT3B AS | NM_006892 | 3′UTR (3993) | PTI-Ome | 5′cgucgtggctccagttacaa3′ | (SEQ ID NO:20) |
| DNMT3B MM | NM_006892 | | PTI-Ome | 5′ccucgtcggtcgacttagaa3′ | (SEQ ID NO:21) |
| DNMT3B AS | NM_006892 | 3′UTR (3023) | PTI | 5′agagctgtcggcactgtggt3′ | (SEQ ID NO:22) |
| DNMT3B AS | NM_006892 | 3′UTR (3023) | PTI-Ome | 5′agagctgtcggcactguggu3′ | (SEQ ID NO:23) |
| DNMT3B MM | NM_006892 | | PTI-Ome | 5′acaggtgtggccagtgucgu3′ | (SEQ ID NO:24) |
| DNMT3B AS | NM_008892 | 3′UTR (3997) | PTI | 5′tgttacgtcgtggctccagt3′ | (SEQ ID NO:25) |
| DNMT3B AS | NM_006892 | 3′UTR (3997) | PTI-Ome | 5′uguuacgtcgtggctccagu3′ | (SEQ ID NO:26) |
| DNMT3B MM | NM_006892 | | PTI-Ome | 5′ucuuaggtcctgcctgcacu3′ | (SEQ ID NO:27) |
| DNMT3A AS | AF067972.1 | 3′UTR (3258) | PTI | 5′tgatgtccaaccctttucgc3′ | (SEQ ID NO:28) |
| DNMT3A AS | AF067972.1 | 3′UTR (3258) | PTI-Ome | 5′ugaugtccaaccctttucgc3′ | (SEQ ID NO:29) |
| DNMT3A AS | AP067972.1 | 3′UTR (3434) | PTI | 5′caggagatgatgtccaaccc3′ | (SEQ ID NO:30) |
| DNMT3A AS | AF067972.1 | 3′UTR (3434) | PTI-Ome | 5′caggagatgatgtccaaccc3′ | (SEQ ID NO:31) |
| DNMT3A MM | AF067972.1 | | PTI-Ome | 5′cacgacatcatctcgaacgc3′ | (SEQ ID NO:32) |
| DNMT3A AS | AF067972.1 | 3′UTR (4045) | PTI | 5′cgtgagaacgcgccatctgc3′ | (SEQ ID NO:33) |
| DNMT3A AS | AF067972.1 | 3′UTR (4045) | PTI-Ome | 5′cgugagaacgcgccatcugc3′ | (SEQ ID NO:34) |
| DNMT3A MM | AF067972.1 | | PTI-Ome | 5′ccugacaaggcccgatgugc3′ | (SEQ ID NO:35) |
| DNMT3A AS | AF067972.1 | 3′UTR (4302) | PTI | 5′gttctgatcccaccacaagg3′ | (SEQ ID NO:36) |
| DNMT3A AS | AF067972.1 | 3′UTR (4302) | PTI-Ome | 5′guuctgatcccaccacaagg3′ | (SEQ ID NO:37) |

PTI refers to a phosphorothioate backbone as opposed to a phosphodiester backbone
PTI-Ome refers to a phosphorothioate backbone with 2′-O-methyl modifications occurring in the first four and last four bases of these oligonucleotides. Where thymine occurs in the oligonucleotide sequence at these positions, it is replaced by uracil.

The antisense oligonucleotides according to the invention may optionally be formulated with any of the well known pharmaceutically acceptable carriers or diluents (see preparation of pharmaceutically acceptable formulations in, e.g., Remington's Pharmaceutical Sciences, 18th Edition, ed. A. Gennaro, Mack Publishing Co., Easton, Pa., 1990), with the proviso that such carriers or diluents not affect their ability to modulate DNA MeTase activity.

By way of non-limiting example, the agent of the first aspect of the invention may also be a small molecule inhibitor. The term “small molecule” as used in reference to the inhibition of DNA MeTase is used to identify a compound having a molecular weight preferably less than 1000 Da, more preferably less than 800 Da, and most preferably less than 600 Da, which is capable of interacting with a DNA MeTase and inhibiting the expression of a nucleic acid molecule encoding an DNMT isoform or activity of an DNMT protein. Inhibiting DNA MeTase enzymatic activity means reducing the ability of a DNA MeTase to add a methyl group to the C5 position of cytosine. In some preferred embodiments, such reduction of DNA MeTase activity is at least about 50%, more preferably at least about 75%, and still more preferably at least about 90%. In other preferred embodiments, DNA MeTase activity is reduced by at least 95% and more preferably by at least 99%. In one certain embodiment, the small molecule inhibitor is an inhibitor of one or more but less than all DNMT isoforms. By “all DNMT isoforms” is meant all proteins that specifically add a methyl group to the C5 position of cytosine, and includes, without limitation, DNMT-1, DNMT3a, or DNMT3b, all of which are considered “related proteins,” as used herein.

Most preferably, a DNA MeTase small molecule inhibitor interacts with and reduces the activity of one or more DNA MeTase isoforms (e.g., DNMT3a and/or DNMT3b), but does not interact with or reduce the activities of all of the other DNA MeTase isoforms (e.g., DNMT-1, DNMT3a and DNMT3b). As discussed below, a preferred DNA MeTase small molecule inhibitor is one that interacts with and reduces the enzymatic activity of a DNA MeTase isoform that is involved in tumorigenesis.

The invention disclosed herein encompasses the use of different libraries for the identification of small molecule inhibitors of one or more, but not all, MeTases. Libraries useful for the purposes of the invention include, but are not limited to, (1) chemical libraries, (2) natural product libraries, and (3) combinatorial libraries comprised of random peptides, oligonucleotides and/or organic molecules.

Chemical libraries consist of structural analogs of known compounds or compounds that are identified as “hits” or “leads” via natural product screening. Natural product libraries are derived from collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see , Cane, D. E., et al., (1998) Science 282:63-68. Combinatorial libraries are composed of large numbers of peptides, oligonucleotides or organic compounds as a mixture. They are relatively easy to prepare by traditional automated synthesis methods, PCR, cloning or proprietary synthetic methods. Of particular interest are peptide and oligonucleotide combinatorial libraries.

More specifically, a combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

For a review of combinatorial chemistry and libraries created therefrom, see Huc, I. and Nguyen, R. (2001) Comb. Chem. High Throughput Screen 4:53-74; Lepre, C. A. (2001) Drug Discov. Today 6:133-140; Peng, S. X. (2000) Biomed. Chromatogr. 14:430-441; Bohm, H. J. and Stahl, M. (2000) Curr. Opin. Chem. Biol. 4:283-286; Barnes, C. and Balasubramanian, S. (2000) Curr. Opin. Chem. Biol. 4:346-350; Lepre, Enjalbal, C., et al., (2000) Mass Septrom Rev. 19:139-161; Hall, D. G., (2000) Nat. Biotechnol. 18:262-262; Lazo, J. S., and Wipf, P. (2000) J. Pharmacol. Exp. Ther. 293:705-709; Houghten, R. A., (2000) Ann. Rev. Pharmacol. Toxicol. 40:273-282; Kobayashi, S. (2000) Curr. Opin. Chem. Biol. (2000) 4:338-345; Kopylov, A. M. and Spiridonova, V. A. (2000) Mol. Biol. (Mosk) 34:1097-1113; Weber, L. (2000) Curr. Opin. Chem. Biol. 4:295-302; Dolle, R. E. (2000) J. Comb. Chem. 2:383-433; Floyd, C. D., et al., (1999) Prog. Med. Chem. 36:91-168; Kundu, B., et al., (1999) Prog. Drug Res. 53:89-156; Cabilly, S. (1999) Mol. Biotechnol. 12:143-148; Lowe, G. (1999) Nat. Prod. Rep. 16:641-651; Dolle, R. E. and Nelson, K. H. (1999) J. Comb. Chem. 1:235-282; Czarnick, A. W. and Keene, J. D. (1998) Curr. Biol. 8:R705-R707; Dolle, R. E. (1998) Mol. Divers. 4:233-256; Myers, P. L., (1997) Curr. Opin. Biotechnol. 8:701-707; and Pluckthun, A. and Cortese, R. (1997) Biol. Chem. 378:443.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Biosciences, Columbia, Md., etc.).

Still other libraries of interest include peptide, protein, peptidomimetic, multiparallel synthetic collection, recombinatorial, and polypeptide libraries.

Small molecule inhibitors of one or more, but not all, MeTases are identified and isolated from the libraries described herein by any method known in the art. Such screening methods include, but are not limited to, functional screening and affinity binding methodologies. In addition, the screening methods utilized for the identification of small molecule inhibitors of one or more, but not all, MeTases include high throughput assays. By way of non-limiting example, Meldal, M. discloses the use of combinatorial solid-phase assays for enzyme activity and inhibition experiments (Meldal, M. (1998) Methods Mol. Biol. 87:51-57), and Dolle, R. E. describes generally the use of combinatorial libraries for the discovery of inhibitors of enzymes (Dolle, R. E. (1997) Mol. Divers. 2:223-236).

By way of non-limiting example, Example 5 below provides a small molecule inhibitor screen encompassed by the invention.

The agents according to the invention are useful as analytical tools and as therapeutic tools, including as gene therapy tools. The invention also provides methods and compositions which may be manipulated and fine-tuned to fit the condition(s) to be treated while producing fewer side effects.

In a second aspect, the invention provides a method for inhibiting one or more, but less than all, DNA MeTase isoforms in a cell comprising contacting the cell with an agent of the first aspect of the invention. By way of non-limiting example, the agent may be an antisense oligonucleotide or a small molecule inhibitor that inhibits the expression of one or more, but less than all, specific DNA MeTase isoforms in the cell.

In certain embodiments, the invention provides a method comprising contacting a cell with an antisense oligonucleotide that inhibits one or more but less than all DNA MeTase isoforms in the cell. Preferably, cell proliferation is inhibited in the contacted cell. Thus, the antisense oligonucleotides according to the invention are useful in therapeutic approaches to human diseases, including benign and malignant neoplasms, by inhibiting cell proliferation in cells contacted with the antisense oligonucleotides. The phrase “inhibiting cell proliferation” is used to denote an ability of a DNA MeTase antisense oligonucleotide or a small molecule DNA MeTase inhibitor (or combination thereof) to retard the growth of cells contacted with the oligonucleotide or small molecule inhibitor, as compared to cells not contacted. Such an assessment of cell proliferation can be made by counting contacted and non-contacted cells using a Coulter Cell Counter (Coulter, Miami, Fla.) or a hemacytometer. Where the cells are in a solid growth (e.g., a solid tumor or organ), such an assessment of cell proliferation can be made by measuring the growth with calipers, and comparing the size of the growth of contacted cells with non-contacted cells. Preferably, the term includes a retardation of cell proliferation that is at least 50% greater than non-contacted cells. More preferably, the term includes a retardation of cell proliferation that is 100% of non-contacted cells (i.e., the contacted cells do not increase in number or size). Most preferably, the term includes a reduction in the number or size of contacted cells, as compared to non-contacted cells. Thus, a DNA MeTase antisense oligonucleotide or a DNA MeTase small molecule inhibitor that inhibits cell proliferation in a contacted cell may induce the contacted cell to undergo growth retardation, to undergo growth arrest, to undergo programmed cell death (i.e., to apoptose), or to undergo necrotic cell death.

Conversely, the phrase “inducing cell proliferation” and similar terms are used to denote the requirement of the presence or enzymatic activity of a specific DNA MeTase isoform for cell proliferation in a normal (i.e., non-neoplastic) cell. Hence, over-expression of a specific DNA MeTase isoform that induces cell proliferation may or may not lead to increased cell proliferation; however, inhibition of a specific DNA MeTase isoform that induces cell proliferation will lead to inhibition of cell proliferation.

The cell proliferation inhibiting ability of the antisense oligonucleotides according to the invention allows the synchronization of a population of a-synchronously growing cells. For example, the antisense oligonucleotides of the invention may be used to arrest a population of non-neoplastic cells grown in vitro in the G1 or G2 phase of the cell cycle. Such synchronization allows, for example, the identification of gene and/or gene products expressed during the G1 or G2 phase of the cell cycle. Such a synchronization of cultured cells may also be useful for testing the efficacy of a new transfection protocol, where transfection efficiency varies and is dependent upon the particular cell cycle phase of the cell to be transfected. Use of the antisense oligonucleotides of the invention allows the synchronization of a population of cells, thereby aiding detection of enhanced transfection efficiency.

The anti-neoplastic utility of the antisense oligonucleotides according to the invention is described in detail elsewhere in this specification.

In yet other preferred embodiments, the cell contacted with a DNA MeTase antisense oligonucleotide is also contacted with a DNA MeTase small molecule inhibitor.

In a few preferred embodiments, the DNA MeTase small molecule inhibitor is operably associated with the antisense oligonucleotide. As mentioned above, the antisense oligonucleotides according to the invention may optionally be formulated with well known pharmaceutically acceptable carriers or diluents. This formulation may further contain one or more one or more additional DNA MeTase antisense oligonucleotide(s), and/or one or more DNA MeTase small molecule inhibitor(s), or it may contain any other pharmacologically active agent.

In a particularly preferred embodiment of the invention, the antisense oligonucleotide is in operable association with a DNA MeTase small molecule inhibitor. The term “operable association” includes any association between the antisense oligonucleotide and the DNA MeTase small molecule inhibitor which allows an antisense oligonucleotide to inhibit the expression of one or more specific DNA MeTase isoform-encoding nucleic acids and allows the DNA MeTase small molecule inhibitor to inhibit specific DNA MeTase isoform enzymatic activity. One or more antisense oligonucleotides of the invention may be operably associated with one or more DNA MeTase small molecule inhibitors. In some preferred embodiments, an antisense oligonucleotide of the invention that targets one particular DNA MeTase isoform (e.g., DNMT-1, DNMT3a, or DNMT3b) is operably associated with a DNA MeTase small molecule inhibitor which targets the same DNA MeTase isoform. A preferred operable association is hydrolyzable. Preferably, the hydrolyzable association is a covalent linkage between the antisense oligonucleotide and the DNA MeTase small molecule inhibitor. Preferably, such covalent linkage is hydrolyzable by esterases and/or amidases. Examples of such hydrolyzable associations are well known in the art. Phosphate esters are particularly preferred.

In certain preferred embodiments, the covalent linkage may be directly between the antisense oligonucleotide and the DNA MeTase small molecule inhibitor so as to integrate the DNA MeTase small molecule inhibitor into the backbone. Alternatively, the covalent linkage may be through an extended structure and may be formed by covalently linking the antisense oligonucleotide to the DNA MeTase small molecule inhibitor through coupling of both the antisense oligonucleotide and the DNA MeTase small molecule inhibitor to a carrier molecule such as a carbohydrate, a peptide or a lipid or a glycolipid. Other preferred operable associations include lipophilic association, such as formation of a liposome containing an antisense oligonucleotide and the DNA MeTase small molecule inhibitor covalently linked to a lipophilic molecule and thus associated with the liposome. Such lipophilic molecules include without limitation phosphotidylcholine, cholesterol, phosphatidylethanolamine, and synthetic neoglycolipids, such as syalyllacNAc-HDPE. In certain preferred embodiments, the operable association may not be a physical association, but simply a simultaneous existence in the body, for example, when the antisense oligonucleotide is associated with one liposome and the small molecule inhibitor is associated with another liposome.

In a third aspect, the invention provides a method for inhibiting neoplastic cell proliferation in an animal comprising administering to an animal having at least one neoplastic cell present in its body a therapeutically effective amount of an agent of the first aspect of the invention. In one certain embodiment, the agent is an antisense oligonucleotide of the first aspect of the invention, and the method further comprises a pharmaceutically acceptable carrier. The antisense oligonucleotide and the pharmaceutically acceptable carrier are administered for a therapeutically effective period of time. Preferably, the animal is a mammal, particularly a domesticated mammal. Most preferably, the animal is a human.

The term “neoplastic cell” is used to denote a cell that shows aberrant cell growth. Preferably, the aberrant cell growth of a neoplastic cell is increased cell growth. A neoplastic cell may be a hyperplastic cell, a cell that shows a lack of contact inhibition of growth in vitro, a benign tumor cell that is incapable of metastasis in vivo, or a cancer cell that is capable of metastases in vivo and that may recur after attempted removal. The term “tumorigenesis” is used to denote the induction of cell proliferation that leads to the development of a neoplastic growth.

The terms “therapeutically effective amount” and “therapeutically effective period of time” are used to denote known treatments at dosages and for periods of time effective to reduce neoplastic cell growth. Preferably, such administration should be parenteral, oral, sublingual, transdermal, topical, intranasal, or intrarectal. When administered systemically, the therapeutic composition is preferably administered at a sufficient dosage to attain a blood level of antisense oligonucleotide from about 0.1 μM to about 10 μM. For localized administration, much lower concentrations than this may be effective, and much higher concentrations may be tolerated. One of skill in the art will appreciate that such therapeutic effect resulting in a lower effective concentration of the DNA MeTase inhibitor may vary considerably depending on the tissue, organ, or the particular animal or patient to be treated according to the invention.

In a preferred embodiment, the therapeutic composition of the invention is administered systemically at a sufficient dosage to attain a blood level of antisense oligonucleotide from about 0.01 μM to about 20 μM. In a particularly preferred embodiment, the therapeutic composition is administered at a sufficient dosage to attain a blood level of antisense oligonucleotide from about 0.05 μM to about 15 μM. In a more preferred embodiment, the blood level of antisense oligonucleotide is from about 0.1 μM to about 10 μM.

For localized administration, much lower concentrations than this may be therapeutically effective. Preferably, a total dosage of antisense oligonucleotide will range from about 0.1 mg to about 200 mg oligonucleotide per kg body weight per day. In a more preferred embodiment, a total dosage of antisense oligonucleotide will range from about 1 mg to about 20 mg oligonucleotide per kg body weight per day. In a most preferred embodiment, a total dosage of antisense oligonucleotide will range from about 1 mg to about 10 mg oligonucleotide per kg body weight per day. In a particularly preferred embodiment, the therapeutically effective amount of a DNA MeTase antisense oligonucleotide is about 5 mg oligonucleotide per kg body weight per day.

In certain preferred embodiments of the third aspect of the invention, the method further comprises administering to the animal a therapeutically effective amount of a DNA MeTase small molecule inhibitor with a pharmaceutically acceptable carrier for a therapeutically effective period of time. In some preferred embodiments, the DNA MeTase small molecule inhibitor is operably associated with the antisense oligonucleotide, as described supra.

The DNA MeTase small molecule inhibitor-containing therapeutic composition of the invention is administered systemically at a sufficient dosage to attain a blood level DNA MeTase small molecule inhibitor from about 0.01 μM to about 10 μM. In a particularly preferred embodiment, the therapeutic composition is administered at a sufficient dosage to attain a blood level of DNA MeTase small molecule inhibitor from about 0.05 μM to about 10 μM. In a more preferred embodiment, the blood level of DNA MeTase small molecule inhibitor is from about 0.1 μM to about 5 μM. For localized administration, much lower concentrations than this may be effective. Preferably, a total dosage of DNA MeTase small molecule inhibitor will range from about 0.01 mg to about 100 mg protein effector per kg body weight per day. In a more preferred embodiment, a total dosage of DNA MeTase small molecule inhibitor will range from about 0.1 mg to about 50 mg protein effector per kg body weight per day. In a most preferred embodiment, a total dosage of DNA MeTase small molecule inhibitor will range from about 0.1 mg to about 10 mg protein effector per kg body weight per day. In a particularly preferred embodiment, the therapeutically effective synergistic amount of DNA MeTase small molecule inhibitor (when administered with an antisense oligonucleotide) is about 5 mg per kg body weight per day.

Certain preferred embodiments of this aspect of the invention result in an improved inhibitory effect, thereby reducing the therapeutically effective concentrations of either or both of the nucleic acid level inhibitor (i.e., antisense oligonucleotide) and the protein level inhibitor (i.e., DNA MeTase small molecule inhibitor) required to obtain a given inhibitory effect as compared to those necessary when either is used individually.

Furthermore, one of skill will appreciate that the therapeutically effective synergistic amount of either the antisense oligonucleotide or the DNA MeTase inhibitor may be lowered or increased by fine tuning and altering the amount of the other component. The invention therefore provides a method to tailor the administration/treatment to the particular exigencies specific to a given animal species or particular patient. Therapeutically effective ranges may be easily determined for example empirically by starting at relatively low amounts and by step-wise increments with concurrent evaluation of inhibition.

In a fourth aspect, the invention provides a method for identifying a specific DNA MeTase isoform that is required for induction of cell proliferation comprising contacting a growing cell with an agent of the first aspect of the invention. In certain preferred embodiments, the agent is an antisense oligonucleotide that inhibits the expression of a DNA MeTase isoform, wherein the antisense oligonucleotide is specific for a particular DNMT isoform, and thus inhibition of cell proliferation in the contacted cell identifies the DNA MeTase isoform as a DNA MeTase isoform that is required for induction of cell proliferation. In other certain embodiments, the agent is a small molecule inhibitor that inhibits the activity of a DNA MeTase isoform, wherein the small molecule inhibitor is specific for a particular DNMT isoform, and thus inhibition of cell proliferation in the contacted cell identifies the DNA MeTase isoform as a DNA MeTase isoform that is required for induction of cell proliferation. In certain preferred embodiments, the cell is a neoplastic cell, and the induction of cell proliferation is tumorigenesis. In still yet other preferred embodiments of the fourth aspect of the invention, the method comprises an agent of the first aspect of the invention which is a combination of one or more antisense oligonucleotides and/or one or more small molecule inhibitors of the first aspect of the invention. In certain preferred embodiments, the DNA MeTase isoform is DNMT-1, DNMT3a, or DNMT3b. In other certain preferred embodiments, the DNA MeTase isoform is DNMT3a and/or DNMT3b.

In a fifth aspect, the invention provides a method for identifying a DNA MeTase isoform that is involved in induction of cell differentiation comprising contacting a cell with an agent that inhibits the expression of a DNA MeTase isoform, wherein induction of differentiation in the contacted cell identifies the DNA MeTase isoform as a DNA MeTase isoform that is involved in induction of cell differentiation. In certain preferred embodiments, the agent is an antisense oligonucleotide of the first aspect of the invention. In other certain preferred embodiments, the agent is an small molecule inhibitor of the first aspect of the invention. In still other certain embodiments, the cell is a neoplastic cell. In still yet other preferred embodiments of the fifth aspect of the invention, the method comprises an agent of the first aspect of the invention which is a combination of one or more antisense oligonucleotides and/or one or more small molecule inhibitors of the first aspect of the invention. In certain preferred embodiments, the DNA MeTase isoform is DNMT-1, DNMT3a, or DNMT3b. In other certain preferred embodiments, the DNA MeTase isoform is DNMT3a and/or DNMT3b.

In a sixth aspect, the invention provides a method for inhibiting neoplastic cell growth in an animal comprising administering to an animal having at least one neoplastic cell present in its body a therapeutically effective amount of an agent of the first aspect of the invention. In certain embodiments thereof, the agent is an antisense oligonucleotide, which is combined with a pharmaceutically acceptable carrier and administered for a therapeutically effective period of time.

In certain embodiments where the agent of the first aspect of the invention is a DNA MeTase small molecule inhibitor, therapeutic compositions of the invention comprising said small molecule inhibitor(s) are administered systemically at a sufficient dosage to attain a blood level DNA MeTase small molecule inhibitor from about 0.01 μM to about 10 μM. In a particularly preferred embodiment, the therapeutic composition is administered at a sufficient dosage to attain a blood level of DNA MeTase small molecule inhibitor from about 0.05 μM to about 10 μM. In a more preferred embodiment, the blood level of DNA MeTase small molecule inhibitor is from about 0.1 μM to about 5 μM. For localized administration, much lower concentrations than this may be effective. Preferably, a total dosage of DNA MeTase small molecule inhibitor will range from about 0.01 mg to about 100 mg protein effector per kg body weight per day. In a more preferred embodiment, a total dosage of DNA MeTase small molecule inhibitor will range from about 0.1 mg to about 50 mg protein effector per kg body weight per day. In a most preferred embodiment, a total dosage of DNA MeTase small molecule inhibitor will range from about 0.1 mg to about 10 mg protein effector per kg body weight per day.

In a seventh aspect, the invention provides a method for investigating the role of a particular DNA MeTase isoform in cellular proliferation, including the proliferation of neoplastic cells. In this method, the cell type of interest is contacted with an amount of an antisense oligonucleotide that inhibits the expression of one or more specific DNA MeTase isoforms, as described for the first aspect according to the invention, resulting in inhibition of expression of DNA MeTase isoform(s) in the cell. If the contacted cell with inhibited expression of the DNA MeTase isoform(s) also shows an inhibition in cell proliferation, then the DNA MeTase isoform(s) is required for the induction of cell proliferation. In this scenario, if the contacted cell is a neoplastic cell, and the contacted neoplastic cell shows an inhibition of cell proliferation, then the DNA MeTase isoform whose expression was inhibited is a DNA MeTase isoform that is required for tumorigenesis. In certain preferred embodiments, the DNA MeTase isoform is DNMT-1, DNMT3a, or DNMT3b. In certain preferred embodiments, the DNA MeTase isoform is DNMT3a and/or DNMT3b.

Thus, by identifying a particular DNA MeTase isoform that is required for in the induction of cell proliferation, only that particular DNA MeTase isoform need be targeted with an antisense oligonucleotide to inhibit cell proliferation or induce differentiation. Consequently, a lower therapeutically effective dose of antisense oligonucleotide may be able to effectively inhibit cell proliferation. Moreover, undesirable side effects of inhibiting all DNA MeTase isoforms may be avoided by specifically inhibiting the one (or more) DNA MeTase isoform(s) required for inducing cell proliferation.

As previously indicated, the agent of the first aspect includes, but is not limited to, oligonucleotides and small molecule inhibitors that inhibit the activity of one or more, but less than all, DNA MeTase isoforms. The measurement of the enzymatic activity of a DNA MeTase isoform can be achieved using known methodologies. For example, see Szyf, M., et al. (1991) J. Biol. Chem. 266:10027-10030.

Preferably, the DNA MeTase small molecule inhibitor(s) of the invention that inhibits a DNA MeTase isoform that is required for induction of cell proliferation is a DNA MeTase small molecule inhibitor that interacts with and reduces the enzymatic activity of fewer than all DNA MeTase isoforms.

In an eighth aspect, the invention provides a method for identifying a DNA MeTase isoform that is involved in induction of cell differentiation, comprising contacting a cell with an antisense oligonucleotide that inhibits the expression of a DNA MeTase isoform, wherein induction of differentiation in the contacted cell identifies the DNA MeTase isoform as a DNA MeTase isoform that is involved in induction of cell differentiation. Preferably, the cell is a neoplastic cell. In certain embodiments, the DNA MeTase isoform is DNMT-1, DNMT3a, or DNMT3b. In certain other embodiments, the DNA MeTase isoform is DNMT3a and/or DNMT3b.

The phrase “inducing cell differentiation” and similar terms are used to denote the ability of a DNA MeTase antisense oligonucleotide or DNA MeTase small molecule inhibitor (or combination thereof) to induce differentiation in a contacted cell as compared to a cell that is not contacted. Thus, a neoplastic cell, when contacted with a DNA MeTase antisense oligonucleotide or DNA MeTase small molecule inhibitor (or both) of the invention, may be induced to differentiate, resulting in the production of a daughter cell that is phylogenetically more advanced than the contacted cell.

In a ninth aspect, the invention provides a method for inhibiting cell proliferation in a cell, comprising contacting a cell with at least two of the agents selected from the group consisting of an antisense oligonucleotide that inhibits a specific DNA MeTase isoform, a DNA MeTase small molecule inhibitor, an antisense oligonucleotide that inhibits a DNA MeTase, and a DNA MeTase small molecule inhibitor. In one embodiment, the inhibition of cell growth of the contacted cell is greater than the inhibition of cell growth of a cell contacted with only one of the agents. In certain preferred embodiments, each of the agents selected from the group is substantially pure. In preferred embodiments, the cell is a neoplastic cell. In yet additional preferred embodiments, the agents selected from the group are operably associated.

In a tenth aspect, the invention provides a method for modulating cell proliferation or differentiation comprising contacting a cell with an agent of the first aspect of the invention, wherein one or more, but less than all, DNA MeTase isoforms are inhibited, which results in a modulation of proliferation or differentiation. In preferred embodiments, the cell proliferation is neoplasia. In certain embodiments, the DNA MeTase isoform is selected from DNMT-1, DNMT3a, and DNMT3b. In certain other embodiments, the DNA MeTase isoform is DNMT3a and/or DNMT3b.

For purposes of this aspect, it is unimportant how the specific DNMT isoform is inhibited. The present invention has provided the discovery that specific individual DNMTs are involved in cell proliferation or differentiation, whereas others are not. As demonstrated in this specification, this is true regardless of how the particular DNMT isoform(s) is/are inhibited.

By the term “modulating” proliferation or differentiation is meant altering by increasing or decreasing the relative amount of proliferation or differentiation when compared to a control cell not contacted with an agent of the first aspect of the invention. Preferably, there is an increase or decrease of about 10% to 100%. More preferably, there is an increase or decrease of about 25% to 100%. Most preferably, there is an increase or decrease of about 50% to 100%. The term “about” is used herein to indicate a variance of as much as 20% over or below the stated numerical values.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein. Such equivalents are considered to be within the scope of this invention, and are covered by the appended claims.

In an eleventh aspect, the invention provides a method for inhibiting cell proliferation in a cell comprising contacting a cell with at least two agents selected from the group consisting of an antisense oligonucleotide from the first aspect of the invention that inhibits expression of a specific DNA MeTase isoform, a small molecule inhibitor that inhibits a specific DNA MeTase isoform, an antisense oligonucleotide that inhibits a histone deactylase, and a small molecule that inhibits a histone deactylase. In one embodiment, the inhibition of cell growth of the contacted cell is greater than the inhibition of cell growth of a cell contacted with only one of the agents. In certain embodiments, each of the agents selected from the group is substantially pure. In preferred embodiments, the cell is a neoplastic cell. In yet additional preferred embodiments, the agents selected from the group are operably associated.

### EXAMPLES

### Example 1

### Synthesis and Identification of Active DNMT3a and DNMT3b Antisense Oligonucleotides

Antisense (AS) were designed to be directed against the 5′- or 3′-untranslated region (UTR) of the targeted genes, DNMT3a and DNMT3b. Oligos were synthesized with the phosphorothioate backbone on an automated synthesizer and purified by preparative reverse-phase HPLC. All oligos used were 20 base pairs in length.

To identify antisense oligodeoxynucleotide (ODN) capable of inhibiting DNMT3a or DNMT3b expression in human cancer cells, antisense oligonucleotides were initially screened in T24 (human blader) A549 (human non small cell lung cancers cells at 100 nM. Cells were harvested after 24 hours of treatment, and DNMT3a or DNMT3b RNA expression was analyzed by Northern blot analysis.

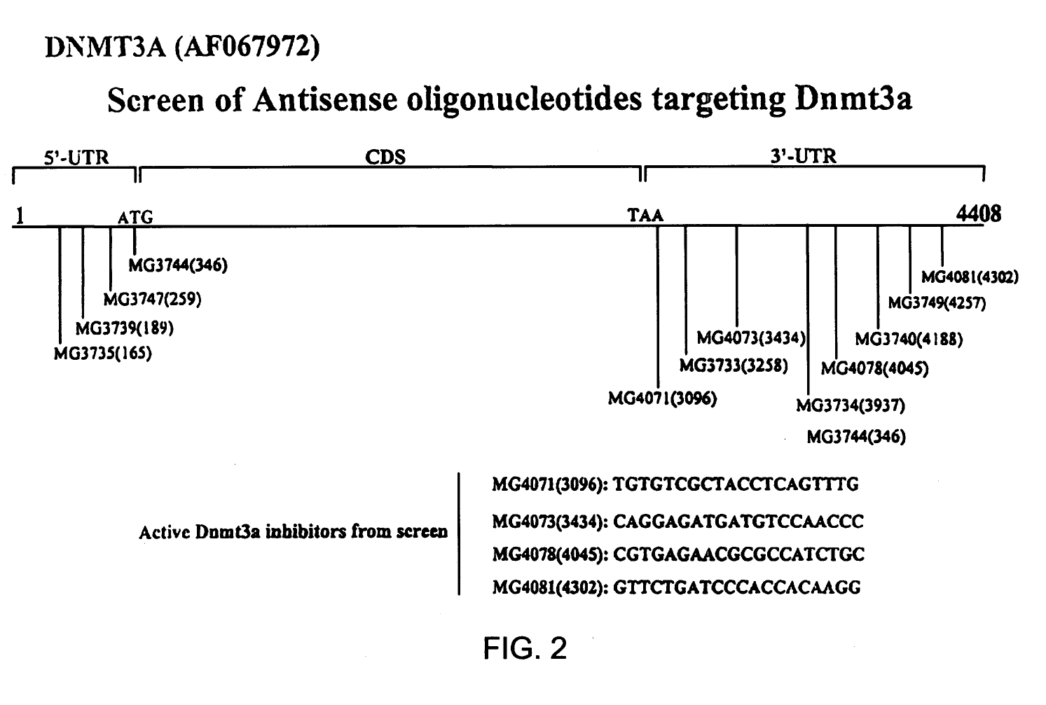

A total of 27 phosphorothioate ODNs containing sequences complementary to the 5′ or 3′ UTR of the human DNMT3a gene (GenBank Accession No. AF067972) were screened as above (FIG. 2). First generation DNMT3a AS-ODNs with greatest antisense activity to human DNMT3a were selected for second generation chemistry production. These oligonucleotides were then synthesized as second generation chemistry (phosphorothioate backbone and 2′-O-methyl modifications) and appropriate mismatch controls of these were prepared.

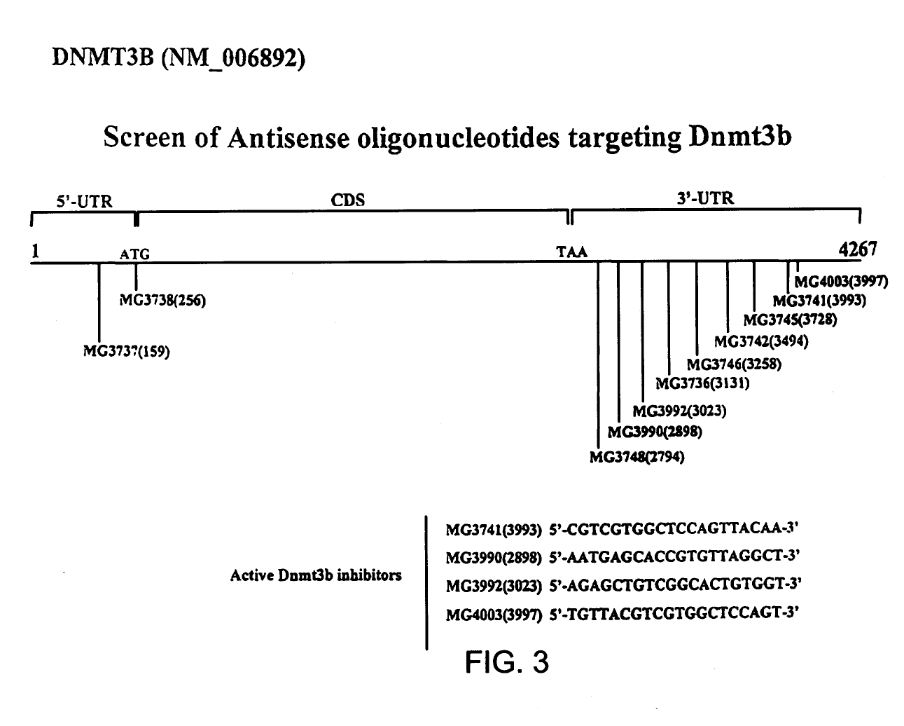

A total of 34 phosphorothioate ODNs containing sequences complementary to the 5′ or 3′ UTR of the human DNMT3b gene (GenBank Accession No. NM_{—}006892) were screened as above (FIG. 3). First generation DNMT3b AS-ODNs with greatest antisense activity to human DNMT3b were selected for second generation chemistry production. These oligonucleotides were then synthesized as second generation chemistry (phosphorothioate backbone and 2′-O-methyl modifications) and appropriate mismatch controls of these were prepared. Table 1 and Table 2 provides a summary of oligonucloetides sequences, nucleotide position, and chemical modifications of antisense oligonucleotides targeting the DNMT1, DNMT3a and DNMT3b genes. Sequences of mismatch control oligonucleotides are also given.

### Example 2

### Dose Dependent Inhibition of DNMT3a and DNMT3b mRNA Expression with Antisense Oligonucleotides

Active oligonucleotides identified in initial screens were then synthesized with phosporothiate backbone modification and 2′-O-methyl modifications of the sugar on the four 5′ and 3′ nucleotides. In order to determine whether AS ODN treatment reduced DNMT3a and DNMT3b expression at the mRNA level dose response experiments were done. Human A549 or T24 cells were treated with increasing doses of antisense (AS) oligonucleotide from 0-75 nM for 24 hours.

Briefly, human A549 or T24 human bladder carcinoma cells were seeded in 10 cm tissue culture dishes one day prior to oligonucleotide treatment. The cell lines were obtained from the American Type Culture Collection (ATCC) (Manassas, Va.) and were grown under the recommended culture conditions. Before the addition of the oligonucleotides, cells were washed with PBS (phosphate buffered saline). Next, lipofectin transfection reagent (GIBCO BRL Mississauga, Ontario, Calif.), at a concentration of 6.25 μg/ml, was added to serum free OPTIMEM medium (GIBCO BRL, Rockville, Md.), which was then added to the cells. The oligonucleotides to be screened were then added directly to the cells (i.e., one oligonucleotide per plate of cells).

Cells were harvested, and total RNAs were analyzed by Northern blot analysis. Briefly, total RNA was extracted using RNeasy miniprep columns (QIAGEN). Ten to twenty μg of total RNA was run on a formaldehyde-containing 1% agarose gel with 0.5 M sodium phosphate (pH 7.0) as the buffer system. RNAs were then transferred to nitrocellulose membranes and hybridized with the radiolabelled DNA probes specific for DNMT3a or DNMT3b messenger RNA. Autoradiography was performed using conventional procedures.

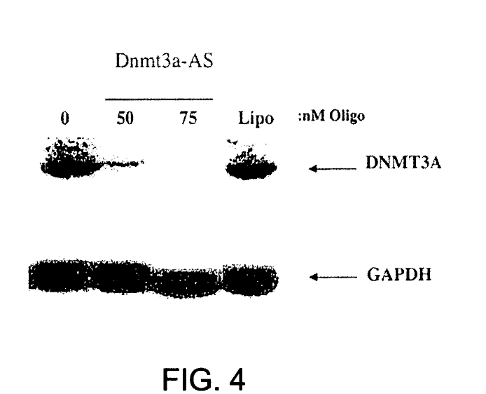

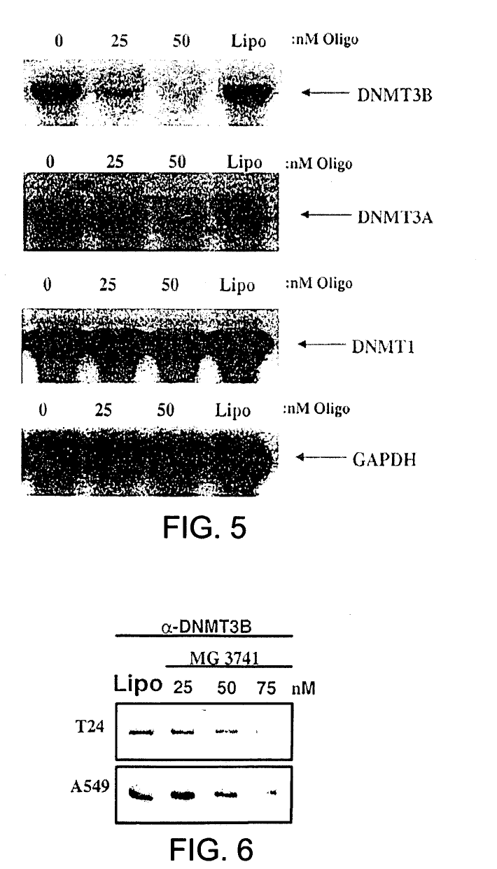

FIG. 4 presents results of experiments done with a first generation antisense inhibitor of DNMT3a. FIG. 5 is a representative Northern blot demonstrating the dose dependent inhibition of DNMT3b expression by AS-ODN (SEQ ID NO: 18) in A549 human non small cell lung cancer cells (estimated IC₅₀ value of 25 nM). Also demonstrated is the specificity of SEQ ID NO: 18 for DNMT3b, as non target mRNAs DNMT1, DNMT3A and Glyceraldehyde 3′-phosphate dehydrogenase are not effected. MM indicates control mismatch oligonucleotides.

Treatment of cells with the indicated AS ODN significantly inhibits the expression of the targeted mRNA DNMT3a and DNMT3b respectively in a dose dependent fashion in both human A549 and T24 cells.

### Example 3

### DNMT3b Antisense ODNs Inhibit DNMT3b Protein Expression

In order to determine whether treatment with DNMT3a or DNMT3b AS-ODNs would inhibit expression at the protein level, antibodies specific for either DNMT3a or DNMT3b were produced for use in western blots. DNMT3b is expressed at sufficiently high levels in human cancer cells to be detected by our DNMT3b antibody. However, DNMT3a is not expressed at detectable levels. Therefore, both human A549 non small cell lung cancer cells and T24 human bladder cancer cells were treated with doses of the DNMT3b antisense inhibitor (SEQ ID NO: 18) ranging from 0-75 nM for 48 hours and then measured DNMT3b protein levels by Western blot.

Briefly, cells were lysed in buffer containing 1% Triton X-100, 0.5% sodium deoxycholate, 5 mM EDTA, 25 mM Tris-HC1, pH 7.5, plus protease inhibitors. Total protein was quantified by the protein assay reagent from Bio-Rad (Hercules, Calif.). 100 ug of total protein was analyzed by SDS-PAGE. Next, total protein was transferred onto a PVDF membrane and probed with DNMT3b specific antibody. Anti-DNMT3b antibody was raised by immunizing rabbits with a GST fusion protein containing a fragment of the DNMT3b protein (amino acids 4-101 of GenBank Accession No. NM_{—}006892). Rabbit antiserum was tested and found only to react specifically to the human DNMT3b isoform. DNMT3b antiserum was used at 1:500 dilution in Western blots to detect DNA MeTase-6 in total cell lysates. Horse Radish Peroxidase conjugated secondary antibody was used at a dilution of 1:5000 to detect primary antibody binding. The secondary antibody binding was visualized by use of the Enhanced chemiluminescence (ECL) detection kit (Amersham-Pharmacia Biotech., Inc., Piscataway, N.J.).

As shown in FIG. 6, the treatment of T24 or A549 cells with DNMT3b AS-ODN MG3741 inhibits the expression of DNMT3b protein.

### Example 4

### Effect of DNMT3a and DNMT3b Inhibition on Cancer Cell Apoptosis and Growth

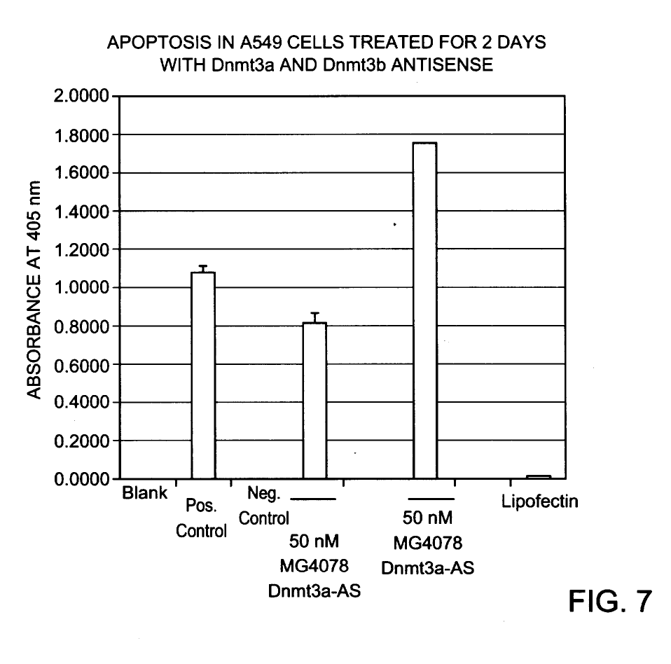

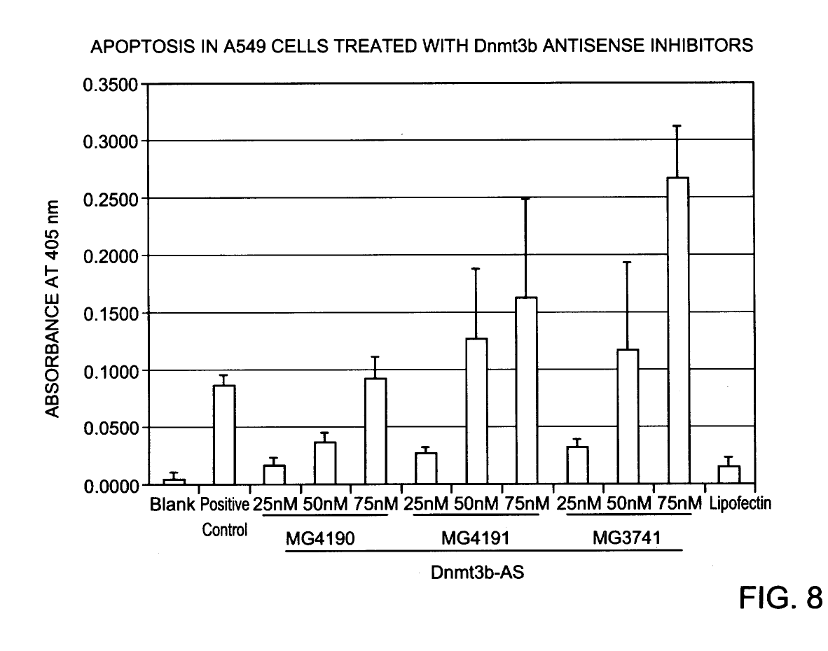

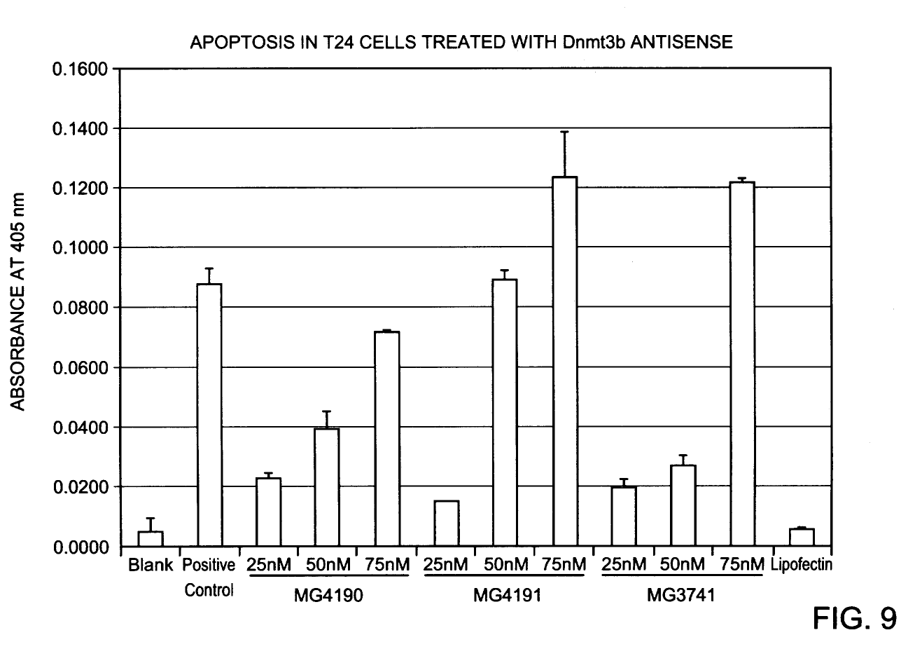

In order to determine the effects of DNMT3a and DNMT3b inhibition on apoptosis of cancer cells, various cancer cell lines (A549 or T24 cells, MDAmb231) were exposed to the DNMT3a and DNMT3b AS-ODN for various periods of time and the effects on apoptosis were determined. For the analysis of apoptosis (active cell death), cells were analyzed using the Cell Death Detection ELISA ^{Plus} kit (Roche Diagnostic GmBH, Mannheim, Germany) according to the manufacturer's directions. Typically, 10,000 cells were plated in 96-well tissue culture dishes for 2 hours before harvest and lysis. Each sample was analyzed in duplicate. ELISA reading was done using a MR700 plate reader (DYNEX Technology, Ashford, Middlesex, England) at 410 nm. The reference was set at 490 nm. Results of these studies on DNMT3a and DNMT3b inhibition in human cancer cells are shown in FIGS. 7-9.

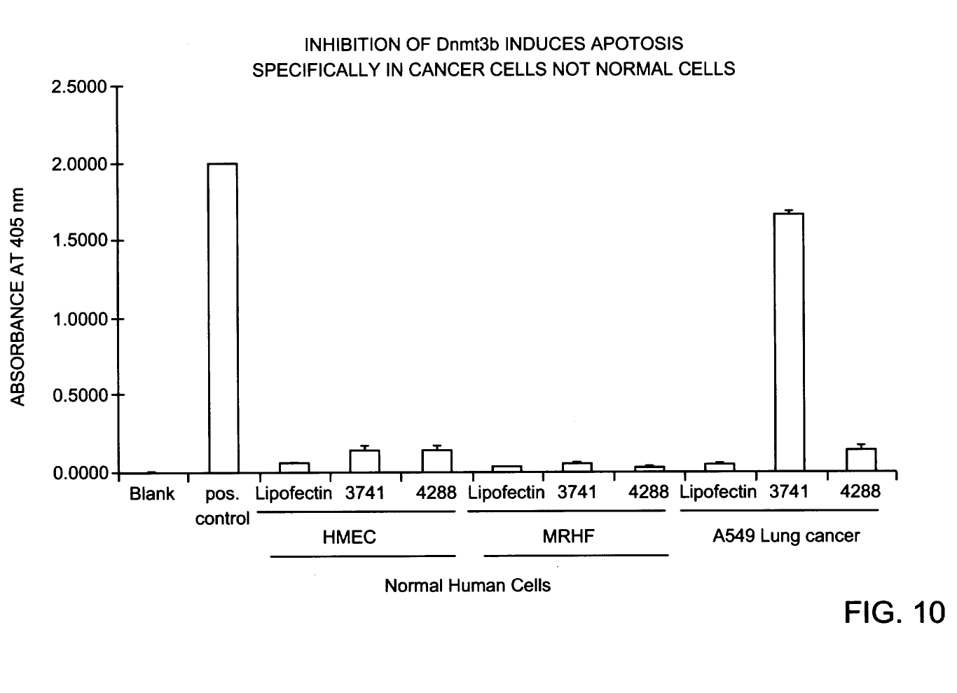

The effect of DNMT3b inhibition on the induction of apoptosis in normal cells was also determined, the results of which are presented in FIG. 10. HMEC (human mammary epithelial cells, ATCC, Manassas, Va.) and MRHF (male foreskin fibroblasts, ATCC, Manassas, Va.) were treated with 75 nM of DNMT3b AS (SEQ ID NO: 18) or its mismatch control SEQ ID NO: 19 for 48 hrs as previously described for human cancer cells. FIG. 10 shows that DNMT3b AS inhibitor does not induce apoptosis in normal cells, but does induces apoptosis in cancer cells.

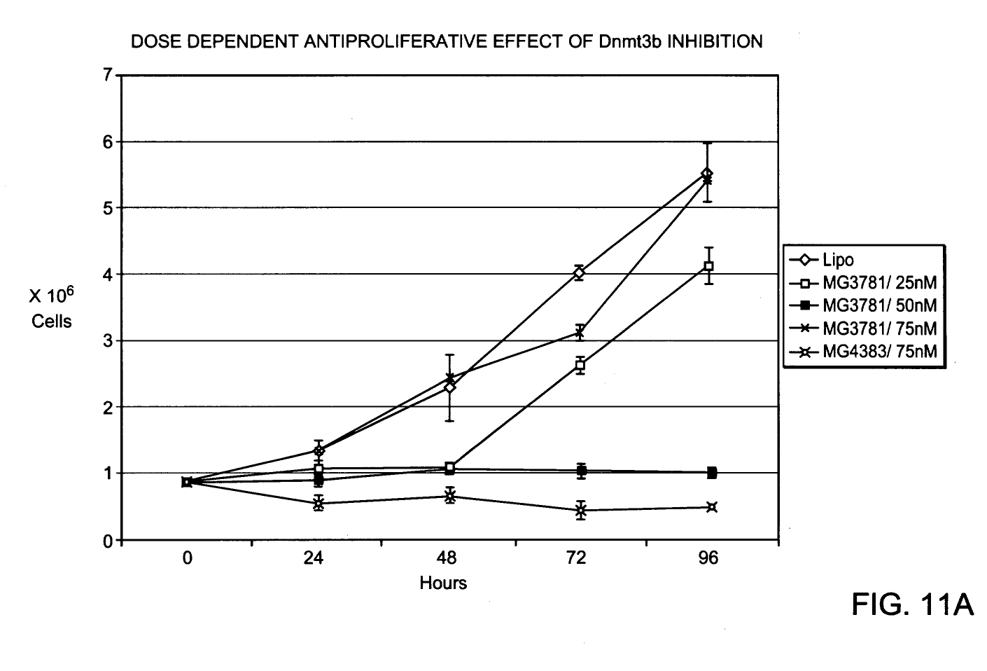

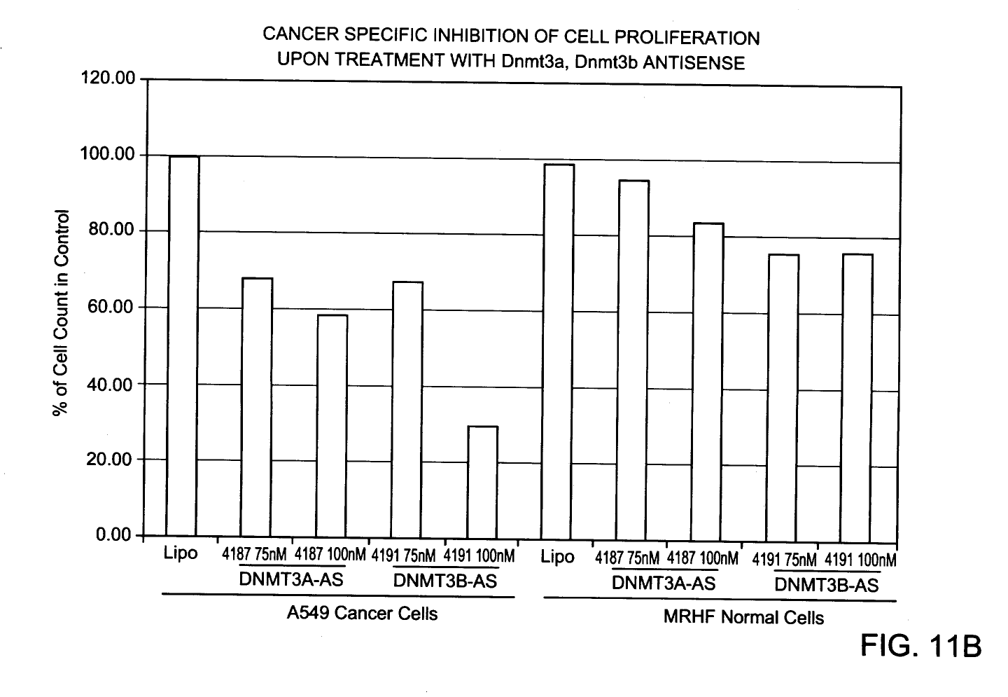

In order to determine the effects of DNMT3a and DNMT3b inhibition on the proliferation of cancer cells, various cancer cell lines (A549 or T24 cells, MDAmb231) were exposed to the DNMT3a and DNMT3b AS-ODN for various periods of time and the effects on cell proliferation were determined. Results of these studies are presented in FIGS. 11A and 11B and demonstrate that the inhibition of DNMT3a or DNMT3b expression dramatically affects cancer cell proliferation.

Results of these studies demonstrate that inhibition of DNMT3a or DNMT3b results in growth inhibition and induces apoptosis of human cancer cells but similar inhibition in normal cells does not. As T24 cells are p53 null whereas A549 cells have functional p53 protein, the induction of apoptosis seen is independent of p53 activity. Taken together these results suggest that inhibition of DNMT3a or DNMT3b may provide specific and effective anticancer therapies.

### Example 5

### Identification of Small Molecule Inhibitors of DNA MethylTransferase Isoforms

DNA methyltransferase enzymatic activity assays and substrate specificity of the various isoforms are performed as described previously (Szyf, M. et al. (1991) J. Biol. Chem. 266:10027-10030). Briefly, Nuclear extracts are prepared from 1×10⁸ mid-log phase human H446 cells or mouse Y1 (ATCC, Manassas, Va.) cells which are grown under standard cell culture conditions. Cells are treated with medium supplemented with the test compound at a concentration of from about 0.001 μM to about 10 mM, or at a concentration of from about 0.01 μM to about 1 mM, or at a concentration of from about 0.1 μM to about 1 mM. The cells are harvested and washed twice with phosphate buffered saline (PBS), then the cell pellet is resuspended in 0.5 ml Buffer A (10 mM Tris pH 8.0, 1.5 mM MgCl₂, 5 mM KCl₂, 0.5 mM DTT, 0.5 mM PMSF and 0.5% Nonidet P40) to separate the nuclei from other cell components. The nuclei are pelleted by centrifugation in an Eppendorf microfuge at 2,000 RPM for 15 min at 4° C. The nuclei are washed once in Buffer A and re-pelleted, then resuspended in 0.5 ml Buffer B (20 mM Tris pH 8.0, 0.25% glycerol, 1.5 mM MgCl₂, 0.5 mM PMSF, 0.2 mM EDTA 0.5 mM DTT and 0.4 mM NaCl). The resuspended nuclei are incubated on ice for 15 minutes then spun at 15,000 RPM to pellet nuclear debris. The nuclear extract in the supernatant is separated from the pellet and used for assays for DNA MeTase activity.

For each assay, carried out in triplicate, 3 μg of nuclear extract is used in a reaction mixture containing 0.1 μg of a synthetic 33-base pair hemimethylated DNA molecule substrate with 0.5 μCi S-[methyl-³ H] adenosyl-L-methionine (78.9 Ci/mmol) as the methyl donor in a buffer containing 20 mM Tris-HCl (pH 7.4), 10 mM EDTA, 25% glycerol, 0.2 mM PMSF, and 20 mM 2-mercaptoethanol. The reaction mixture is incubated for 1 hour at 37° C. to measure the initial rate of the DNA MeTase activity. The reaction is stopped by adding 10% TCA to precipitate the DNA, then the samples are incubated at 4° C. for 1 hour and the TCA precipitates are washed through GFC filters (Fischer, Hampton, N.H.). Controls are DNA incubated in the reaction mixture in the absence of nuclear extract, and nuclear extract incubated in the reaction mixture in the absence of DNA.

The filters are laid in scintillation vials containing 5 ml of scintillation cocktail, and tritiated methyl groups incorporated into the DNA are counted in a scintillation counter according to standard methods. To measure inhibition of DNA MeTase expression, the specific activity of the nuclear extract from test compound-treated cells is compared with the specific activity of the extract from untreated cells. Treatment of cells with test compounds that are candidate small molecule inhibitors of DNA MeTase activity will result in a reduction in DNA MeTase activity in the nuclear extract.

The above assay may be easily adapted for testing the affect of test compounds on the activity of individual, recombinantly produced, DNA MeTase isoforms. In order to produce recombinant protein for each DNA MeTase isoform, an expression construct was produced for each isotype (Dnmt1, Dnmt3a and Dnmt3b (Dnmt3b2 and Dnmt3b3 splice variants)) by inserting the entire coding sequence of the respective isotype into the pBlueBac4.5™ baculovirus expression vector(Invitrogen, Carlsbad, Calif.). Each construct was then used to infect High Five insect cells according to Invitrogen's baculovirus expression manual.

Purification of baculovirus expressed human Dnmt1, Dnmt3a and Dnmt3b proteins was done as follows: Nuclear extract was isolated from High Five insect cells, the salt concentration was adjusted with buffer (20 mM Tris pH 7.4, 1 mM Na₂EDTA, 10% sucrose) to get a final concentration of 0.1M NaCl. Lysate was centrifuged at 9500 g for 10 min. Supernatant was applied to Q-sepharose, Heparin, and Source Q15 column sequentially. All purifications are performed on a gradifrac system with a P1 pump at 4° C.

DNA MeTase isotype specific activity assays are performed according to the following procedure. From about 100 pg to about 25 μg, or more preferably from about 10 ng to about 10 μg, or most preferably from about 100 ng to about 2.5 μg of recombinant DNA MeTase isotype protein is incubated in a reaction mixture containing 0.1 μg of a synthetic 33-base pair hemimethylated DNA molecule substrate with 0.5 μCi S-[methyl-³ H] adenosyl-L-methionine (78.9 Ci/mmol) as the methyl donor in a buffer containing 20 mM Tris-HCl (pH 7.4), 10 mM EDTA, 25% glycerol, 0.2 mM PMSF, and 20 mM 2-mercaptoethanol in a total volume of 30 μl. Test sample also includes the test small molecule inhibitor compound at a concentration of from about 0.001 μM to about 10 mM, or at a concentration of from about 0.01 μM to about 1 mM, or at a concentration of from about 0.1 μM to about 1 mM. The reactions are stopped and the samples are processed as described herein above.

It is expected that certain candidate small molecule inhibitors of DNA MeTase activity will have the affect of significantly decreasing the amount of radioactive methyl incorporated into the substrate DNA.

### EQUIVALENTS

Those skilled in the art will recognize, or be able to ascertain, using no more than routine experimentation, many equivalents to the specific embodiments of the invention described herein. Such equivalents are intended to be encompassed by the following claims.

What is claimed is

1. An agent that inhibits one or more specific DNA methyltransferase isoforms, but less than all DNA methyltransferase isoforms, wherein the agent is selected from the group consisting of an anti-DNA methyltransferase oligonucleotide and a small molecule inhibitor of DNA methyltransferase.

2. The agent according to claim 1 that is an oligonucleotide.

3. The oligonucleotide according to claim 2, wherein the oligonucleotide is a chimeric oligonucleotide.

4. The oligonucleotide according to claim 2, wherein the oligonucleotide is a hybrid oligonucleotide.

5. The oligonucleotide according to claim 2, wherein the oligonucleotide is complementary to a region of RNA or double-stranded DNA selected from the group consisting of
(a) a nucleic acid molecule encoding at least 13 contiguous oligonucleotides from DNMT-1 (SEQ ID NO: 1),
(b) a nucleic acid molecule encoding at least 13 contiguous oligonucleotides from DNMT3a (SEQ ID NO: 2), and
(c) a nucleic acid molecule encoding at least 13 contiguous oligonucleotides from DNMT3b (SEQ ID NO: 3).

6. The oligonucleotide according to claim 5 having a nucleotide sequence of from about 13 to about 35 nucleotides.

7. The oligonucleotide according to claim 5 having a nucleotide sequence of from about 15 to about 26 nucleotides.

8. The oligonucleotide according to claim 5 having one or more phosphorothioate internucleoside linkage, being 20-26 nucleotides in length, and being modified such that the terminal four nucleotides at the 5′ end of the oligonucleotide and the terminal four nucleotides at the 3′ end of the oligonucleotide each have 2′-O-methyl groups attached to their sugar residues.

9. The oligonucleotide according to claim 5, wherein the oligonucleotide is complementary to a region of RNA or double-stranded DNA encoding a portion of DNMT1 (SEQ ID NO: 1).

10. The oligonucleotide according to claim 5 that is selected from the group consisting of SEQ ID NO: 4, SEQ ID NO: 5, SEQ ID NO: 6, SEQ ID NO: 7, SEQ ID NO: 8, SEQ ID NO: 9, and SEQ ID NO: 10.

11. The oligonucleotide according to claim 5, wherein the oligonucleotide is complementary to a region of RNA or double-stranded DNA encoding a portion of DNMT3a (SEQ ID NO: 1).

12. The oligonucleotide according to claim 11 that is selected from the group consisting of SEQ ID NO: 28, SEQ ID NO: 29, SEQ ID NO: 30, SEQ ID NO: 31, SEQ ID NO: 33, SEQ ID NO: 34, and SEQ ID NO: 36.

13. The oligonucleotide according to claim 5, wherein the oligonucleotide is complementary to a region of RNA or double stranded DNA encoding a portion of DNMT3b (SEQ ID NO: 3).

14. The oligonucleotide according to claim 13 that is selected from the group consisting of SEQ ID NO: 18, SEQ ID NO: 20, SEQ ID NO: 21, SEQ ID NO: 22, SEQ ID NO: 23, SEQ ID NO: 25, SEQ ID NO: 26, and SEQ ID NO: 27.

15. A method for inhibiting one or more DNA methyltransferase isoforms in a cell comprising contacting the cell with the agent according to claim 1.

16. A method for inhibiting one or more DNA methyltransferase isoforms in a cell comprising contacting the cell with the oligonucleotide according to claim 2.

17. The method according to claim 16, wherein cell proliferation is inhibited in the contacted cell.

18. The method according to claim 16, wherein the oligonucleotide that inhibits cell proliferation in a contacted cell induces the contacted cell to undergo growth retardation.

19. The method according to claim 16, wherein the oligonucleotide that inhibits cell proliferation in a contacted cell induces the contacted cell to undergo growth arrest.

20. The method according to claim 16, wherein the oligonucleotide that inhibits cell proliferation in a contacted cell induces the contacted cell to undergo programmed cell death.

21. The method according to claim 16, wherein the oligonucleotide that inhibits cell proliferation in a contacted cell induces the contacted cell to undergo necrotic cell death.

22. The method according to claim 16, further comprising contacting the cell with a DNA methyltransferase small molecule inhibitor.

23. A method for inhibiting neoplastic cell proliferation in an animal comprising administering to an animal having at least one neoplastic cell present in its body a therapeutically effective amount of the agent of claim 1.

24. A method for inhibiting neoplastic cell proliferation in an animal comprising administering to an animal having at least one neoplastic cell present in its body a therapeutically effective amount of the oligonucleotide of claim 2.

25. The method according to claim 24, wherein the animal is a human.

26. The method according to claim 24, further comprising administering to the animal a therapeutically effective amount of a DNA methyltransferase small molecule inhibitor with a pharmaceutically acceptable carrier for a therapeutically effective period of time.

27. The method according to claim 26, wherein the animal is a human.

28. A method for identifying a DNA methyltransferase isoform that is required for the induction of cell proliferation, the method comprising contacting the DNA methyltransferase isoform with an inhibitory agent, wherein a decrease in the induction of cell proliferation indicates that the DNA methyltransferase isoform is required for the induction of cell proliferation.

29. The method according to claim 28, wherein the inhibitory agent is an oligonucleotide of claim 2.

30. A method for identifying a DNA methyltransferase isoform that is required for cell proliferation, the method comprising contacting the DNA methyltransferase isoform with an inhibitory agent, wherein a decrease in cell proliferation indicates that the DNA methyltransferase isoform is required for cell proliferation.

31. The method according to claim 30, wherein the inhibitory agent is an oligonucleotide of claim 2.

32. A method for identifying a DNA methyltransferase isoform that is required for the induction of cell differentiation, the method comprising contacting the DNA methyltransferase isoform with an inhibitory agent, wherein an induction of cell differentiation indicates that the DNA methyltransferase isoform is required for the induction of cell proliferation.

33. The method according to claim 32, wherein the inhibitory agent is an oligonucleotide of claim 2.

34. A method for inhibiting cell proliferation in a cell, comprising contacting a cell with at least two agents selected from the group consisting of an antisense oligonucleotide that inhibits a specific DNA methyltransferase isoform, a DNA methyltransferase small molecule inhibitor that inhibits a specific DNA methyltransferase isoform, an antisense oligonucleotide that inhibits a DNA methyltransferase, and a DNA methyltransferase small molecule inhibitor.

35. A method for modulating cell proliferation or differentiation of a cell comprising inhibiting a specific DNA methyltransferase isoform that is involved in cell proliferation or differentiation by contacting the cell with an agent of claim 1.

36. The method according to claim 35, wherein the cell proliferation is neoplasia.

37. The method according to claim 36, wherein the DNA methyltransferase isoform is selected from the group consisting of DNMT-1, DNMT3a and DNMT3b.

38. The method according to claim 37, wherein the DNA methyltransferase isoform is selected from DNMT3a and DNMT3b.

39. The method according to claim 37, wherein the DNA methyltransferase is DNMT3b.
